(19) United States
(12) Patent Application Publication
(10) Pub. No.: US 2012/0100185 A1
(43) Pub. Date: Apr. 26, 2012

(54) REGENERATION OF TISSUE WITHOUT CELL TRANSPLANTATION

(76) Inventors: Xuejun Wen, Mount Pleasant, SC (US); Yongzhi Qiu, Charleston, SC (US); Wendy S. Vanden Berg-Foels, Mount Pleasant, SC (US)

(21) Appl. No.: 13/263,232

(22) PCT Filed: Apr. 13, 2010
(86) PCT No.: PCT/US10/30865

§ 371 Date: Dec. 1, 2011

Related U.S. Application Data
(60) Provisional application No. 61/168,769, filed on Apr. 13, 2009.

(51) Int. Cl. A61K 38/14; A61K 38/30; A61K 38/22; A61K 38/20; C07K 17/10; A61P 43/00; C08H 1/00; A61K 9/14; A61P 19/04; B01J 19/12; A61K 38/18; C07K 2/00; B82Y 5/00

(52) U.S. Cl. 424/400; 514/16.5; 514/8.8; 514/8.9; 514/8.5; 514/8.6; 514/9.5; 514/7.6; 514/8.2; 514/8.7; 424/85.2; 530/351; 530/395; 514/9.1; 514/9.6; 530/354; 525/54.1; 530/397; 204/157.68; 977/773; 977/915

(57) ABSTRACT

The present invention provides methods and compositions for tissue regeneration without cell transplantation.

### STATEMENT OF PRIORITY

This application claims the benefit, under 35 U.S.C. §119(e), of U.S. Provisional Application No. 61/168,769, filed Apr. 13, 2009, the contents of which are incorporated by reference herein in their entirety.

### FEDERAL SUPPORT OF THE INVENTION

Aspects of this invention were funded under Grant No. EPS-0447660 of the National Science Foundation USA. The U.S. Government has certain rights in this invention.

### FIELD OF THE INVENTION

The present invention relates to compositions and methods for tissue regeneration without cell transplantation.

### BACKGROUND OF THE INVENTION

Articular cartilage does not mount an effective repair response to injury, resulting in progressive joint degeneration and patient disability. The prevalence of clinical osteoarthritis in the United States was 27 million in 2005¹, with over 21 million physician visits incurred². For patients with focal cartilage injury/damage, there are several treatment options. One option is anti-inflammatory medication (e.g., systemic or local administration of steroids and other anti-inflammation medicine, cryotherapy, etc.), which only decreases the symptoms but does not treat the underlying disease. Another option is artificial joint fluid injections (e.g., hyaluronic acid injection), which also do not treat the underlying disease. A further option is surgical resurfacing (such as chondroplasty and microfracture) to remove the unstable cartilage and stimulate the underlying bone to bleed and form clots which will form fibrocartilage later. Replacement (e.g., autologous or allogenic graft replacement) is an additional treatment option. A further option is the GENZYME CARTICELL® implant system (Autologous Chondrocyte Implantation, ACI) and finally there is joint replacement. In 2006, approximately 773,000 people in the United States received a total hip or knee replacement^{3,4}; the number is projected to be over four million in the year 2030⁵. However, joint replacement is not suitable for younger patients or patients with earlier stages of degeneration. For the CARTICELL® approach, two surgical procedures are needed, which are painful and expensive. Although the first surgery can be done using minimal intervention, such as arthroscopic surgery, the second surgery is an open joint procedure. Although other new methods use scaffolds as carriers for cell transplantation, these methods share the same problems as the CARTICELL® method.

Because cartilage does not mount a successful repair response, healing of a defect must be engineered. Wound healing for most tissues begins with an inflammation stage in which platelets form a clot at a lacerated vessel and release chemoattractants for inflammatory cells¹⁷. Because cartilage is avascular, platelet clotting is not available to initiate an inflammatory stage. Macrophages, one of the first cell types to migrate to the wound, release a variety of growth factors that orchestrate the healing stages. Subsequent stages include chemotaxis of tissue-specific cells and mesenchymal stem cells (MSC), cell proliferation and differentiation, and finally strengthening of the new tissue. However, simple administration of growth factors to the site of a defect does not result in healing because the half-life of most growth factors is less than two hours in vivo^{18,19}.

Tissue engineering strategies to regenerate articular cartilage have included use of autologous cell transplantation, biodegradable scaffolds, and bioactive molecule (biomolecule) delivery. For autologous transplantation therapy, cells are harvested, expanded in in vitro culture, and implanted into the cartilage defect^{6,7}. In vitro cell expansion is contraindicated in patients sensitive to the antibiotics and bovine products used in cell culture⁸. This approach also requires additional procedures for the patient and introduces economic as well as regulatory issues. Subchondral drilling and microfracture in the defect have been used to introduce endogenous bone marrow MSC (BMSC)⁹. Patients often experience a temporary reduction in symptoms after these procedures; however, the fibrocartilage that is generated is mechanically inferior to articular cartilage and degrades rapidly^{9,10}. Further, simple cell delivery, with or with out in vitro expansion, is not a successful strategy for cartilage defect healing.

The only treatment approved by the Food and Drug Administration (FDA) for cartilage defects is autologous chondrocyte transplantation⁶. In this procedure, cartilage is harvested from the joint margin, chondrocytes are expanded in in vitro culture, and the cells are implanted in the defect. The cells are held in place by a periosteum flap sutured to surrounding cartilage. This approach has several drawbacks, including a second surgery, scarcity of harvest sites, potential harvest site morbidity, potential immune response to traces of antibiotics and bovine products used in cell culture, difficulty in suturing the periosteal flap, frequent flap loosening, and significant economic cost^{10,20-22}. Drilling and microfracture of subchondral bone in the defect have been used to promote clot formation and provide access to BMSC beneath the subchondral bone⁹. The fibrocartilage generated by both of these procedures offers temporary symptomatic relief, but does not produce long-term durable hyaline cartilage.

Due to the lack of practical and long-term solutions for repairing injury or damage to cartilage, methods for regeneration of durable articular cartilage without cell transplantation are urgently needed. The present invention overcomes previous shortcomings in the art by providing compositions and methods of their use in regenerating tissue without cell transplantation.

### SUMMARY OF THE INVENTION

The present invention is directed to compositions and methods for tissue regeneration without cell transplantation.

Accordingly, one aspect of the invention is a biocompatible, biodegradable, elastic scaffold comprising one or more than one biomolecule of this invention for regenerating tissue in a subject, with the proviso that the scaffold is cell-free at the time of implantation of the scaffold into the body.

Furthermore, one aspect of the invention is a biocompatible, biodegradable, three-dimensional scaffold comprising a photocurable polysaccharide (e.g., photocurable chitosan) and a protein (e.g., gelatin).

A further aspect of the invention is a method of producing a scaffold comprising photocurable polysaccharide and protein, comprising: a) adding a photocurable polysaccharide in a solvent to a protein-solvent mixture to make a polysaccharide-protein-solvent mixture; b) adding a photoinitiator to the mixture of step (a) above; and c) exposing the polysaccharide-protein-DMSO mixture of step (b) to light to photocure the photocurable polysaccharide, whereby a scaffold comprising photocurable polysaccharide and protein is produced.

A further aspect of the invention is a method of regenerating tissue in a subject, comprising contacting the subject with a scaffold of the present invention comprising one or more biomolecules of this invention. In some aspects of the invention, the subject does not receive a cell transplantation prior to, in conjunction with, or after contacting with the scaffold. In other aspects of the invention, the subject does not receive a cell transplantation in conjunction with the scaffold. Thus, in particular aspects of the invention, the subject does not receive exogenous cells in conjunction with the scaffold.

A further aspect of the invention is a method of regenerating cartilage in a subject (e.g., in a subject having a partial cartilage defect; full thickness defect and/or osteochondral defect), comprising contacting the defect with a scaffold of the present invention under conditions whereby cartilage is regenerated in a subject.

### BRIEF DESCRIPTION OF THE FIGURES

FIGS. 1A-I show schematic diagrams of hypothesized structural/phase changes in a chitosan-gelatin-dimethyl sulfoxide (DMSO) system as a function of setting time (t) before and after ultraviolet (UV) exposure. FIGS. 1A, 1B and 1C show the system before UV exposure as a function of the setting time; FIGS. 1D, 1E and 1F show the system after UV exposure as a function of the setting time; FIGS. 1G, 1H and 1I show the system after EDC crosslinking of gelatin, and gelatin with chitosan as a function of the setting time. The dotted line circles the DMSO (solvent)-rich phase in the gelatin-chitosan complex coacervate. Coils represent gelatin molecules. Rods represent chitosan molecules. Small circles represent DMSO molecules.

FIGS. 2A-I show representative scanning electron microscope (SEM) images of the hybrid scaffolds (5% gelatin-5% chitosan) made with different setting times and with or without crosslinking of gelatin. FIGS. 2A, 2B and 2C show the hybrid scaffolds without boiling water treatment and no crosslinking of gelatin; FIGS. 2D, 2E and 2F show the hybrid scaffolds with boiling water treatment and no crosslinking of gelatin; FIGS. 2G, 2H and 2I show the hybrid scaffolds with no boiling water treatment and with crosslinking of gelatin; FIGS. 2A, 2D and 2G show the hybrid scaffolds with no setting time; FIGS. 2B, 2E and 2H show the hybrid scaffolds with a 8 hour setting time; and FIGS. 2C, 2F and 2I show the hybrid scaffolds with a 12 hour setting time.

FIGS. 3A-L show representative SEM images of the hybrid scaffolds (5% gelatin-7.5% chitosan) made with different setting times and with or without crosslinking of gelatin. FIGS. 3A, 3B, 3C and 3D show scaffolds with no boiling water treatment and no crosslinking of gelatin; FIGS. 3E, 3F, 3G and 3H show scaffolds with boiling water treatment and no crosslinking of gelatin; FIGS. 3I, 3J, 3K and 3L show scaffolds with no boiling water treatment and with crosslinking of gelatin; FIGS. 3A, 3E and 3I show scaffolds with no setting time; FIGS. 3B, 3F and 3J show the scaffolds with a 8 hour setting time; FIGS. 3C, 3G and 3K3 show the scaffolds with a 24 hour setting time; FIGS. 3D, 3H and 3L, show the scaffolds with a 48 hour setting time.

FIGS. 4A-D show representative SEM images of the surfaces (FIGS. 4A-B) and inner structures (FIGS. 4C-D) of the gelatin-chitosan scaffolds (5% gelatin-5% chitosan with 0 setting time) with nanostructures, such as gelatin beads (FIG. 4B) and nanopores (FIG. 4D).

FIG. 5 shows measurements of the storage modulus of the hybrid scaffolds containing different ratios of gelatin to chitosan and with different setting times.

FIGS. 6A-D show a compression test on the chitosan-gelatin hybrid scaffolds (5%-5%, 0 setting time) using a Dynamic Mechanical Analyzer Q800 (DMAQ800). FIG. 6A shows the initial stage of the compression test; FIG. 6B shows the late stage of the compression test with strain close to 90%; FIG. 6C shows the scaffold before the test; and FIG. 6D shows the fully recovered scaffold after the test.

FIGS. 7A-B show the strain-stress curve of the chitosan-gelatin hybrid scaffolds (5%-5%, 0 setting time) during the static compression test. FIG. 7A shows a full range of compression up to 90% strain; and FIG. 7B shows amplification of the curve at low strains ranging from 0 to 50%. Region (a) of FIG. 7B indicates the linear elasticity (bending); region (b) of FIG. 7B indicates the plateau (elastic bucking); and region (c) of FIG. 7B indicates the densification of the scaffold.

FIGS. 8A-D show a cyclic compression test on the chitosan-gelatin hybrid scaffolds (5%-5%, 0 setting time) at a constant strain rate of 1 mm/min and a strain range of 30% to 60%. FIG. 8A shows static force vs. time; FIG. 8B shows strain vs. time; FIG. 8C shows stress vs. time; and FIG. 8D shows stress-strain curve.

FIG. 9 is a confocal image of osteoblasts cultured on the scaffold of a 5% gelatin-7.5% chitosan hybrid scaffold (no setting time and no crosslinking of gelatin) at 48 hours. Osteoblasts were stained with Alexa-488 conjugated phalloidin, and nuclei were stained with Draq-5.

FIGS. 10A-C show the assessment of the multipotency of the expanded synovial cells using standard in vitro assays for chondrogenesic (FIG. 10A, Safranin O fast green), osteogenesic (FIG. 10B, von Kossa), and adipogenesic (FIG. 10C, oil red O) differentiation.

FIGS. 11A-B provide the results of a flow cytometry assay. FIG. 11A shows 99.7% of the gated cells were positive for CD44 with only 2.2% also positive for CD14; and FIG. 11B shows 17.7% of the CD44 positive cells were also CD90 positive.

FIGS. 12A-D show SEM images of 5% gelatin-5% chitosan scaffolds with an eight hr setting time. FIG. 12A shows macrostructure; FIG. 12B shows the pore interior; FIG. 12C shows gelatin beads on the pore surface; and FIG. 12D shows nanopores.

FIG. 13A shows the release of BMP-2 from a thiolated HA-collagen-fibronectin hydrogel over a 10 week period in vitro. FIG. 13B shows the effect of immobilized heparin on the controlled release of HGF from HA-gelatin hydrogels in vitro.

FIGS. 14A-B show high-resolution SEM images of prolyl hydroxylase inhibitor (PHI)-loaded (FIG. 14A) microspheres and (FIG. 14B) nanoparticles. FIG. 14C shows the evaluation of PHI release kinetics from the nanoparticles over a three week period of time.

FIGS. 15A-B show confocal laser microscope images of ECM-based hydrogels with (FIG. 15A) and without (FIG. 15B) HGF, which were implanted subcutaneously on the back of a mouse one week earlier. FIG. 15C shows the number of cells in the hydrogel and FIG. 15D shows the number of stro-1 positive MSC.

FIGS. 16A-B show a schematic of a biomolecule delivery method (FIG. 16A) and temporal release pattern (FIG. 16B).

FIGS. 17 A1-B3 show cartilage defect healing six weeks post implantation with cell-free highly elastic scaffolds encoded with temporal multiple growth factor delivery. FIGS. 17A1-A2: Control scaffolds with only IGF-1 delivery. FIGS. 17B1-B3: Treatment scaffolds with temporal multiple growth factor delivery. FIGS. 17B2-B3: Safranin 0 stain in red showing hyaline cartilage regeneration at the lesion site.

FIGS. 18A-B show cartilage defect healing six weeks post implantation with cell-free highly elastic scaffolds encoded with temporal multiple growth factor delivery. FIG. 18A: Control scaffolds with only IGF-1 delivery. FIG. 18B: Treatment scaffolds with temporal multiple growth factor delivery. Dark (brown) staining for Collagen type II showing hyaline cartilage regeneration at the lesion site.

FIGS. 19A-B show cartilage defect healing six weeks post implantation with cell-free highly elastic scaffolds encoded with temporal multiple growth factor delivery. FIG. 19A: Control scaffolds with only IGF-1 delivery. FIG. 19B: Treatment scaffolds with temporal multiple growth factor delivery. Lack of staining (brown) for collagen type I indicating hyaline cartilage regeneration at the lesion site.

### DETAILED DESCRIPTION

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a biomolecule or agent of this invention, dose, time, temperature, and the like, is meant to encompass variations of ±20%, ±10%, ±5%, ±1%, ±0.5%, or even ±0.1% of the specified amount.

As used herein, the term “consists essentially of” (and grammatical variants) means that the immunogenic composition of this invention comprises no other material immunogenic agent other than the indicated agent(s). The term “consists essentially of” does not exclude the presence of other components such as adjuvants, immunomodulators, and the like.

As used herein, “osteochondral defect” includes any type of damage, injury, disease or disorder (e.g., age-related disorder) in cartilage and/or the bone associated with the cartilage.

The present invention provides a new strategy for repair of tissue damage without cell transplantation. Specifically, the present invention provides methods of regenerating tissue in a subject in the absence of cell transplantation, by delivering to the subject a scaffold of this invention that promotes tissue regeneration (e.g., via recruitment and/or activation of endogenous stem cells to the site of regeneration). Thus, in one embodiment, the present invention provides a one-step process for tissue regeneration in a subject wherein a highly elastic, biocompatible scaffold comprising one or more biomolecules (e.g., growth factors) is contacted with the subject at a site where tissue regeneration is needed and/or desired. Such biomolecules and/or growth factors can be delivered to cue endogenous stem cells for mobilization and migration, proliferation and/or functional differentiation (e.g., chondrogenesis). Endogenous stem cells can be recruited into the scaffold first, which then proliferate and differentiate into the desired cell type(s). In embodiments in which cartilage repair is the desired type of tissue regeneration, endogenous stem cells from synovium membrane and underlying bone can be recruited into the scaffold first, which then proliferate and differentiate into chondrocytes. Thus, the spatio-temporal biomolecule/growth factor delivery system using biocompatible nanoparticles, hydrogels, and scaffolds can mimic the events and/or stages of normal tissue healing.

Thus, one aspect of the present invention is a biocompatible, biodegradable, three-dimensional, cell-free scaffold comprising one or more biomolecules of this invention attached, linked, held within and/or bound to the scaffold. The biomolecule or biomolecules of this invention can be present in any combination in and/or associated in any combination with any biodegradable elastic scaffold in addition to those exemplified herein. For example, a scaffold (e.g., a cell free scaffold) of this invention can comprise, consist essentially of and/or consist of collagen (e.g., collagen I, collagen II, collagen IV), polycation poly(allylanion hydrochloride) (PAH), polyanion (polyacrylic acid) (PAA), polycation poly(styrene sulfonate) (PSS), poly(lactic-co-glycolic acid) (PLGA), polyglycolide, poly(glycolide-co-caprolactone), poly(glycolide-co-trimethylene carbonate), polycaprolactone (PCL), polyurethane (PU), polypropylene carbonate, polyglycolic acid, polyhydroxybutyrate (e.g., poly-3-hydroxybutyrate), polylactic acid, polydioxanone, chitosan, laminin, glycosaminoglycan (e.g., hyaluronic acid), proteoglycan, heparin, elastin, fibrin, fibronectin, chondroitin sulphate proteoglycan, thiolated collagen, thiolated laminin; thiolated fibronectin, thiolated heparin, thiolated hyaluronic acid, thiolated hyaluronan-collagen-fibronectin, cellulose, gelatin and any combination thereof.

In some embodiments, the scaffold of this invention (e.g., a cell free scaffold) can be treated with a crosslinking and/or catalyzing agent [e.g., 1-ethyl-(3-3-dimethylaminopropyl carbodiimide hydrochloride (EDC); N,N′-dicyclohexylcarbodiimide (DCC); N,N′-diisoproplycarbodiimide (DIC), genipin and any other crosslinking and/or catalyzing agent known in the art for crosslinking proteins, in any combination]. In certain embodiments of the methods of this invention, the scaffold is crosslinked with genipin.

Non-limiting examples of non-toxic, elastic, biodegradable scaffolds of the present invention include the scaffolds selected from the group consisting of: (1) chitosan and gelatin; (2) chitosan and collagen; (3) chitosan, collagen, gelatin; (4) elastin; (5) elastin and collagen; (6) elastin and chitosan; (7) polyurethane; (8) poly(lactide-co-caprolactone); (9) poly(glycolide-co-caprolactone); (10) poly(1,8-octanediol citrate); (11) polydimethylsiloxane; (11) gelatin and Poly(lactide-co-caprolactone); (12) polyurea; and the like.

Thus, a further aspect of the present invention is a biocompatible, biodegradable, three-dimensional scaffold comprising a photocurable polysaccharide and a protein. The scaffold provides a 3-dimensional (3D), porous, inter-connected surface for nutrient diffusion and migration, adhesion, proliferation, and/or chondrogenic differentiation of recruited stem cells²⁸. The scaffold further provides mechanical support for the new tissue and degrades as the cells generate extracellular matrix (ECM).

In the production of a scaffold of the present invention, the polysaccharides and proteins can interact through a variety of mechanisms involving van de Waals force, hydrophobic interactions, electrostatic interactions, hydrogen bonding, and/or covalent bindings¹. These interactions make it possible to create polysaccharide-protein complexes with unique physical and morphological properties for biomedical applications¹. In solution, polysaccharide-protein-solvent interactions can lead to phase separation². Depending upon the affinity between the polysaccharide, the protein, and the solvent, segregative phase separation or complex coacervation results³. Complex coacervation (also termed associative phase separation) occurs when the interactions between the polysaccharide and the protein are weakly attractive and non-specific, giving rise to soluble or insoluble polysaccharide-protein complexes⁴. During a coacervation process, a homogenous solution of charged polysaccharide and protein molecules undergoes liquid-liquid phase separation, with the polysaccharide and the protein concentrated in one phase (the biopolymer-rich, solvent-poor phase, or the coacervate), and the solvent enriched in the other phase (the solvent-rich, biopolymer-poor phase). These two liquid phases are not directly miscible, but are strongly interacting. The phase separation in coacervation is driven by the electrostatic and solute-solvent interactions. Due to the smaller sizes and better motility of the solvent molecules when compared to those of the polysaccharide and the protein, the solvent molecules tend to infiltrate into the biopolymer-rich (polysaccharide-protein) phase over time; a process associated with an overall entropy gain of the system. As a result, the polysaccharide-protein complex coacervation is transient and reversible. Evolution takes place in the system with the eventual disappearance of the phase separation, forming one homogeneous phase. By governing the transient phase separation process in the polysaccharide-protein-organic solvent system, it is possible to fabricate three-dimensional porous scaffolds with tunable microstructures across the nano, micro and macro length scales, as well as mechanical properties superior to most existing natural biopolymers, and excellent cytocompatibility.

In some embodiments of the invention, the photocurable polysaccharide of the present invention can be, but is not limited to, chitosan, hyaluronic acid, dextran, alginate, cellulose and any other photocurable polysaccharide now known or later identified. In particular embodiments of the invention, the photocurable polysaccharide of the scaffold is photocurable chitosan.

Chitosan is a naturally-abundant biodegradable linear-cationic polysaccharide that can be produced by partial deacetylation of chitin derived from naturally occurring crustacean shells. Chitosan has a structure similar to that of extracellular matrix (ECM) glycosaminoglycan (GAG)^{33,34}. It is biocompatible, bioadhesive, intrinsically antibacterial, biodegrades in a predictable manner, and is easily processed³⁴. Chitosan has been shown to accelerate healing of skin wounds³³ and to stimulate both osteogenesis and chondrogenesis³⁵.

Chitosan can be chemically modified through substitutions of the hydroxyl groups in the side chains with benzoic groups and methacrylate groups. The incorporation of benzoic groups in the side chains of the modified chitosan improves its solubility in organic solvents (e.g., dimethyl sulfoxide (DMSO)), while the presence of methacrylate groups imparts light curability. The modified photocurable chitosan retains its cationic property and is readily soluble in DMSO. Upon exposure to an irradiation source that initiates or activates the curing process (e.g., ultraviolet light, visible light), the modified chitosan undergoes curing with microscopic changes through chain crosslinking, as well as macroscopic changes as a result of converting from a liquid form into a solid phase.

Thus, another aspect of the present invention provides a photocurable chitosan comprising benzoic groups and methacrylate groups substituted for the chitosan side chain hydroxyl groups. Nonlimiting examples of a water-soluble photocurable chitosan of this invention include styrenated chitosan (Matsuda et al., Biomacromolecules 3(5):942-950 (2002)) and Az-CH-LA (Ishihara et al., Biomaterials 23(s):833-840 (2002)). The entire contents of these references are incorporated herein.

In some aspects of the invention, the protein in the scaffold can be, but is not limited to gelatin, collagen, elastin, laminin, fibronectin and any other protein or peptide that could be combined with the photocurable polysaccharide of this invention to form an elastic, biodegradable, biocompatible scaffold as described herein. In particular aspects of the invention, the protein of the scaffold is gelatin.

Gelatin is a polyampholyte naturally derived from denatured collagen. Like many other proteins, it has a heterogeneous charge distribution on the surface with the presence of both negatively charged and positively charged patches⁶. The peptide sequence of gelatin facilitates cell attachment and proliferation⁷. Gelatin scaffolds have been shown to promote chondrogenic differentiation in bone marrow stem cells (BMSC)³⁰ and adipose-derived mesenchymal stem cells (MSC)³¹. Adding gelatin to a composite scaffold has been shown to increase type II collagen expression by BMSC in vitro³².

In still further embodiments of the invention, the scaffold can comprise photocurable chitosan and gelatin. Studies on chitosan-gelatin interactions and the fabrication of chitosan-gelatin composite scaffolds^{6, 14-16} have generally concluded that the interactions between chitosan and gelatin are electrostatic in nature (ionic strength-dependent)⁶. Strong attractive interactions may occur between negatively charged patches on gelatin and positively charged chitosan. In comparison, interactions between biopolymers (gelatin or chitosan) and organic solvents are usually weak and non-specific. For example, gelatin may interact with DMSO via hydrogen bonding, while chemically modified chitosan with DMSO through hydrophobic interactions. Thus, by governing the chitosan-gelatin interactions and complexation in organic solvent systems, hybrid scaffolds of chitosan and gelatin can be produced with tunable microstructures and properties that are useful for tissue regeneration.

A further aspect of the invention is biomolecule delivery to a subject via a scaffold of this invention, e.g., at a site where tissue regeneration is needed and/or desired. Biomolecule delivery requirements are to be taken into account when selecting materials for scaffold fabrication. Both the method of biomolecule incorporation method and the degradation rate of the biomaterial will determine the release kinetics of the biomolecule. Temporal release features to be considered include the ability to end delivery of the biomolecule after a period of time, to delay the onset of delivery, and/or to generate a sustained release. The cationic property of polysaccharides, such as chitosan, results in electrostatic interactions with negatively charged molecules, including glycoaminoglycan (GAG) and many growth factors³⁴. Many cytokines and growth factors are linked to GAG (primarily with heparin and heparin sulphate), therefore in some embodiments, a scaffold material similar to GAG, and one that also binds GAG, is desirable to retain and concentrate growth factors produced by colonizing cells³⁴. This interaction can be exploited to protect growth factor biologic activity and prolong delivery to the defect site^{38,39}.

Furthermore, short-term biomolecule and/or signal delivery can be achieved by encapsulating the biomolecule in nanospheres and/or microspheres, the production and use of which are well-known in the art. Nanoparticles and microspheres can be delivered to the subject via a scaffold of the present invention or can be delivered directly to the subject. Material selection for the nanoparticle and microsphere diameter will determine the length of the biomolecule delivery period. Additionally, biomolecule delivery corresponding to cell infiltration can be achieved, e.g., by using an enzymatically sensitive hydrogel³⁷.

Cueing mesenchymal stem cells (MSC) to mobilize, migrate, proliferate, and/or differentiate is key to engineering a tissue regeneration and/or healing response in tissues, such as, for example, cartilage. Sources of MSC include bone marrow, periostium, and adipose tissue. Recently, the synovial membrane was also shown to be a rich source of MSC⁴² with superior chondrogenic potential^{43,44}. There are many biomolecules, particularly growth factors, which play a role in cartilage development and regeneration. Candidates for engineering the healing cascade include members of the bone morphogenic protein (BMP) family known to regulate cell fate determination and promote chondrogenesis and osteogenesis¹⁵. BMPs with potential for cartilage regeneration include BMP-2, BMP-4, BMP-5, BMP-6, and BMP-7. BMP-4 and BMP-7 are particularly promising. BMP-4 induces chondrogenic maturation of MSC, suppresses hypertrophy, and stimulates type II collagen and aggrecan production¹⁵. BMP-7 upregulates chondrocyte metabolism and protein synthesis. Culture of MSC with bFGF promotes maintenance of multipotency⁴⁵ and chemotaxis⁴⁶. Hepatocyte growth factor⁴⁷ and stromal cell-derived factor −1⁴⁸ have both been reported to have a strong chemotaxic effect on MSC. Platelet derived growth factor is a mitogenic and chemotactic factor for cells of mesenchymal origin⁴⁹. Transforming growth factor β-1 and β-3 are known to induce and maintain the chondrogenic phenotype¹⁶. Production of extracellular matrix (ECM) is promoted and hypertrophy is inhibited. Insulin-like growth factor −I and −II stimulate directed migration in bone-marrow-derived MSC⁴⁶. Insulin-like growth factor 1 also stimulates proteoglycan production in a dose-dependent manner⁴⁹. Interleukin 10 has immunosuppression activity and may inhibit the migration of macrophages to the defect site⁵⁰. MSC migrate when stimulated with interleukin 8⁵¹. Biomolecules of the present invention can be present as a protein or biologically active peptide thereof or in the form of a nucleic acid encoding the biomolecule protein or biologically active peptide thereof.

Accordingly, in some embodiments, the scaffold of the present invention can be used for biomolecule delivery to a subject of this invention. In further embodiments, the biomolecules in the form of proteins, peptides and/or nucleic acids can be delivered directly to the subject. Biomolecules in the form of proteins, peptides and/or nucleic acids can be incorporated into the scaffold at any step in the fabrication of the scaffold. Thus, the biomolecule can be incorporated at a pre-fabrication step, during fabrication or post-fabrication. Therefore, biomolecules can be attached to separate component of a scaffold prior to fabrication (e.g., attached to the polysaccharide pre-fabrication) or biomolecules can be attached to and/or immobilized on the surface of the scaffold and/or incorporated into the scaffold prior to and/or after curing. In some embodiments of the invention, at least one biomolecule is bound directly (i.e., without any linking or intervening material) to the scaffold. Biomolecules can be attached directly to the scaffold via, for example, physical electrostatic force, wherein the negative charges in the biomolecule(s) bind with the positive charges in the polysaccharide (e.g., chitosan). Biomolecules can also be attached directly to the scaffold via chemically covalent binding by EDC chemistry. Biomolecules with carboxyl groups, such as protein and heparin, can react with the polysaccharide (e.g., chitosan) through the amino acid side groups by EDC chemistry. A further example of direct binding of biomolecules to the scaffold is via chemical crosslinking such as photocrosslinking. Biomolecules with photocurable groups can be co-cross-linked with the photocurable polysaccharide.

In other embodiments, at least one biomolecule can be bound to the scaffold through a linking molecule (i.e., a molecule attached at one site to the biomolecule and attached at a different site to the scaffold). Linking molecules of the invention include, but are not limited to, heparin and heparin sulphate. In particular embodiments of the invention, at least one biomolecule is bound to the scaffold through heparin. In embodiments in which heparin is used as a linking molecule, biomolecules can be used that bind to the heparin by electrostatic force or specific binding. For example, heparin has specific binding with TGF-B1, IL-10, HGF, FGF and others, as is well known in the art. Furthermore, heparin is negatively charged and can bind positively charged biomolecules via electrostatic forces. Additional linking molecules of this invention include heparin analogs and modified polysaccharides, e.g., as described in Frank et al. (J. Biol. Chem. 278(44):43229-43235 (2003)).

In some embodiments, the biomolecules of this invention can be attached to the scaffold directly and/or via a linking molecule in any proportion and/or combination. For example, the same biomolecule can be attached to the scaffold both directly and via a linking molecule and/or multiple biomolecules can be attached to the scaffold in a configuration such that some biomolecules are attached directly and other biomolecules are attached via a linking molecule. Furthermore, more than one linking molecule can be used in the same scaffold, in any combination. Thus, the present invention further comprises embodiments wherein some biomolecules are bound directly to the scaffold and some biomolecules are bound to the scaffold via a linking molecule. The biomolecules attached to the scaffold directly and/or via a linking molecule can be the same biomolecule or different biomolecules in any combination and in any ratio or percentage relative to one another.

A biomolecule of the present invention includes, but is not limited to, a differentiation stimulating biomolecule, a chemotaxis stimulating molecule, a proliferation stimulating biomolecule, a mobilization stimulating biomolecule, or any combination thereof.

Thus, non-limiting examples of biomolecules of present invention include autocrine motility factor, bone morphogenetic proteins (BMPs), epidermal growth factor (EGF), erythropoietin (EPO), fibroblast growth factor (e.g., FGF, FGF-4, FGF-6, FGF-7, FGF-8, FGF-9, FGF-10, FGF-16, FGF-17, FGF-18, FGF-19, FGF-20, FGF-21, FGF-23, FGF-acidic, FGF-basic, HBGF-1, HBGF-2, HBGF-4, HBGF-5, HBGF-6, HBGF-7, KGF-2, and the like), granulocyte-colony stimulating factor (G-CSF), granulocyte-macrophage colony stimulating factor (GM-CSF), growth differentiation factor-9 (GDF9), hepatocyte growth factor (HGF), hepatoma derived growth factor (HDGF), migration-stimulating factor (MSF), nerve growth factor (e.g., NGF, β-NFG, and the like) and other neurotrophins (e.g, NTF-3, NTF-4, and the like), activin (e.g., activin A, activin B, FRP, and the like), thrombopoietin (TPO), vascular endothelial growth factor (VEGF), placental growth factor (P1GF), interleukin (e.g., IL-1, IL-2, IL-3, IL-4, IL-5, IL-6, 11-7, IL-8, IL-9, IL-10, IL-11, IL-12, IL-13, IL-14, IL-15, IL-16, IL-17, IL-18, IL-19, IL-20, IL-21, and the like), interferon (e.g., IFN, IFN-α2a, IFN-α2b, IFN-β, IFN-β1a, IFN-β1b IFN-γ, and the like), B cell activating factor (e.g., TNFSF13B, BLys), β-defensin 2, β-defensin 3, cardiotrophin (CT-1), galectin (e.g., galectin-1, galectin-3, and the like), growth regulated oncogene (e.g., CXCL1, CXCL2, and the like), insulin-like growth factor (e.g., IGF, IGF-I, IGF-II, and the like), insulin-like growth factor binding protein (e.g., IGFBP, IGFBP-1, IGFBP-2, IGFBP-3, IGFBP-4, IGFBP-5, IGFBP-6, IGFBP-7, and the like), monocyte chemotactic protein (e.g., CCL2, MCAF, and the like), macrophage colony-stimulating factor, macrophage inflammatory protein, platelet-derived growth factor (e.g., PDGF, PDGF-AA, PDGF-AB, PDGF-BB, and the like), stem cell factor and stem cell growth factor (e.g., SCF, MGF, SCGF-α, SCGF-β, and the like), stromal cell derived factor, transforming growth factor (e.g., TGF-α, TGF-β), myostatin (GDF-8), tumor necrosis factor (e.g., TNF, TNF-α, TNF-β, TNFSF2, and the like), and any combination thereof.

In some embodiments of the invention, the differentiation stimulating biomolecule includes, but is not limited to, a bone morphogenic protein (BMP, including BMP-1, BMP-2, BMP-4, BMP-5, BMP-6, BMP-7, BMP-8a and/or BMP-9), a transforming growth factor (TGF), including TGF-alpha, TGF-beta 1, TGF-beta 2 and TGF-beta 3, vitamin B12, an insulin-like growth factor-I (e.g., IGF-I; Stem Cells 22:1152-1167 (2004)), IGF-II, or any combination thereof.

In other embodiments, the chemotaxis and/or proliferation stimulating biomolecule includes, but is not limited to, a hepatocyte growth factor (HGF), a stromal cell-derived growth factor-1 (SDF-1), a platelet derived growth factor-bb (PDGF-bb), an insulin-like growth factor (IGF), including IGF-I and IGF-II, an insulin-like growth factor binding protein (IGFBP), including IGFBP-1, IGFBP-2, IGFBP-3, IGFBP-4, IGFBP-5, IGFBP-6, IGFBP-7, TGF-beta 1, TGF-beta 3, BMP 2, BMP 4, BMP 7, basic fibroblast growth factor (bFGF) an interleukin (e.g., interleukin-8; interleukin-10) or any combination thereof.

In further embodiments of the invention, the mobilization stimulating biomolecule includes, but is not limited to, a hepatocyte growth factor (HGF), a stromal cell-derived growth factor-1 (SDF-1), a platelet derived growth factor-bb (PDGF-bb), an insulin-like growth factor (IGF), including IGF-I and IGF-II, an insulin-like growth factor binding protein (IGFBP), including IGFBP-1, IGFBP-2, IGFBP-3, IGFBP-4, IGFBP-5, IGFBP-6, IGFBP-7, TGF-beta 1, TGF-beta 3, BMP 2, BMP 4, BMP 7, basic fibroblast growth factor (bFGF), FGF, EGF, an interleukin (e.g., interleukin-8; interleukin-10) or any combination thereof.

In still further embodiments, the bone morphogenic protein (BMP) includes, but is not limited to, BMP-2, BMP-4, BMP-5, BMP-6, BMP-7, or any combination thereof. In yet other aspects of the invention, the transforming growth factor (TGF) includes, but is not limited to, TGF β-1, TGF β-3, or any combination thereof. In other aspects of the invention, the insulin-like growth factor (IGF) includes, but is not limited to, IGF-I, IGF-II, or any combination thereof. Thus, in particular aspects of the invention, the differentiation stimulating biomolecule that is an insulin-like growth factor is IGF-1. In other aspects of the invention, the chemotaxis and/or proliferation stimulating biomolecule that is an insulin-like growth factor is IGF-1, IGF-2, or any combination thereof. In further embodiments, the insulin-like growth factor binding protein (IGFBP) includes but is not limited to IGFBP-3, IGFBP-5, or any combination thereof. In still further embodiments, the interleukin is selected from the group consisting of IL-8, IL-10, or any combination thereof.

In further embodiments, a biomolecule is provided to a subject in a concentration of about 1 μg to about 10 mg. Thus, in some embodiments of the present invention, a biomolecule is provided to a subject in a concentration range of about 1 μg to about 5 μg, about 1 μg to about 10 μg, about 1 μg to about 15 μg, about 1 μg to about 20 μg, about 1 μg to about 25 μg, about 1 μg to about 30 μg, about 1 μg to about 35 μg, about 1 μg to about 40 μg, about 1 μg to about 50 μg, about 1 μg to about 60 μg, about 1 μg to about 70 μg, about 1 μg to about 80 μg, about 1 μg to about 90 μg, about 1 μg to about 100 μg, about 10 μg to about 20 μg, about 10 μg to about 40 μg, about 10 μg to about 50 μg, about 10 μg to about 60 μg, about 10 μg to about 80 μg, about 10 μg to about 100 μg, about 50 μg to about 100 μg, about 50 μg to about 200 μg, about 50 μg to about 400 μg, about 50 μg to about 500 μg, about 100 μg to about 200 μg, about 100 μg to about 400 μg, about 100 μg to about 600 μg, about 100 μg to about 1000 μg, about 1 μg to about 1 mg, about 1 μg to about 2 mg, about 1 μg to about 3 mg, about 1 μg to about 4 mg, about 1 μg to about 5 mg, about 1 μg to about 6 mg, about 1 μg to about 7 mg, about 1 μg to about 8 mg, about 1 μg to about 9 mg, about 5 μg to about 1 μg, about 5 μg to about 2 mg, about 5 μg to about 4 mg, about 5 μg to about 6 mg, about 5 μg to about 8 mg, about 5 μg to about 10 mg, about 10 μg to about 1 mg, about 10 μg to about 2 mg, about 10 μg to about 4 mg, about 10 μg to about 6 mg, about 10 μg to about 8 mg, about 10 μg to about 10 mg, about 20 μg to about 1 mg, about 20 μg to about 2 mg, about 20 μg to about 4 mg, about 20 μg to about 6 mg, about 20 μg to about 8 mg, about 20 μg to about 10 mg, about 50 μg to about 1 mg, about 50 μg to about 2 mg, about 50 μg to about 4 mg, about 50 μg to about 6 mg, about 50 μg to about 8 mg, about 50 μg to about 10 mg, about 100 μg to about 1 mg, about 100 μg to about 2 mg, about 100 μg to about 4 mg, about 100 μg to about 6 mg, about 100 μg to about 8 mg, about 100 μg to about 10 mg, about 250 μg to about 1 mg, about 250 μg to about 2 mg, about 250 μg to about 4 mg, about 250 μg to about 6 mg, about 250 μg to about 8 mg, about 250 μg to about 10 mg, about 500 m to about 1 mg, about 500 μg to about 2 mg, about 500 μg to about 4 mg, about 500 μg to about 6 mg, about 500 m to about 8 mg, about 500 μg to about 10 mg, about 1 mg to about 2 mg, about 1 mg to about 3 mg, about 1 mg to about 4 mg, about 1 mg to about 5 mg, about 1 mg to about 6 mg, about 1 mg to about 8 mg, about 1 mg to about 10 mg, and the like.

In further embodiments, the a biomolecule is provided to a subject in a concentration of about 2 μg, 3 μg, 4 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 70 μg, 80 μg, 90 μg, 100 μg, 200 μg, 300 μg, 400 μg, 500 μg, 600 μg, 700 μg, 800 μg, 900 μg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, and the like. As one of skill in the art would understand, when more than one biomolecule is provided to a subject, the concentration of the more than one biomolecules can be the same or different from one another.

In particular embodiments of the present invention, the one or more biomolecules delivered to a subject of this invention can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

Non-limiting examples of elastic, non-toxic, biocompatible, biodegradable scaffolds of the present invention can comprise, consist essentially of and/or consist of scaffolds selected from the group consisting of: (1) chitosan and gelatin; (2) chitosan and collagen; (3) chitosan, collagen, and gelatin; (4) elastin; (5) elastin and collagen; (6) elastin and chitosan; (7) polyurethane; (8) poly(lactide-co-caprolactone); (9) poly(glycolide-co-caprolactone); (10) poly(1,8-octanediol citrate); (11) polydimethylsiloxane; (11) gelatin and Poly(lactide-co-caprolactone); (12) polyurea; and any combination thereof.

Thus, in some embodiments of the present invention, the elastic, biodegradable scaffold can comprise, consist essentially of and/or consist of chitosan and gelatin and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In other embodiments of the invention, the elastic, biodegradable scaffold can comprise, consist essentially of and/or consist of chitosan and collagen and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In still other embodiments of the invention, the elastic, biodegradable scaffold can comprise, consist essentially of and/or consist of chitosan, collagen, and gelatin and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In additional embodiments of the present invention, the elastic, biodegradable scaffold can comprise, consist essentially of and/or consist of elastin and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In some embodiments of the present invention, the elastic, biodegradable scaffold can comprise, consist essentially of and/or consist of elastin and collagen and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In further embodiments of the present invention, the elastic, biodegradable scaffold can comprise, consist essentially of and/or consist of elastin and chitosan and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In some embodiments of the present invention, the elastic, biodegradable scaffold can comprise, consist essentially of and/or consist of polyurethane and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In other embodiments of the present invention, the elastic, biodegradable scaffolds can comprise, consist essentially of and/or consist of poly(lactide-co-caprolactone) and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2:

In further embodiments of the invention, the elastic, biodegradable scaffolds can comprise, consist essentially of and/or consist of poly(glycolide-co-caprolactone) and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In additional embodiments of the present invention, the elastic, biodegradable scaffolds can comprise, consist essentially of and/or consist of poly(1,8-octanediol citrate) and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In some embodiments of the present invention, the elastic, biodegradable scaffold can comprise, consist essentially of and/or consist of polydimethylsiloxane and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In other embodiments of the present invention, the elastic, biodegradable scaffold can comprise, consist essentially of and/or consist of gelatin and poly(lactide-co-caprolactone) and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In additional embodiments of the present invention, the elastic, biodegradable scaffold can comprise, consist essentially of and/or consist of polyurea and the one or more biomolecules delivered to a subject can comprise, consist essentially of and/or consist of a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.

In some embodiments of this invention, a hydrogel can be included in the scaffold, e.g., for long term delivery of biomolecules both in vitro and in vivo. Thus, in some embodiments of the invention, the scaffold further comprises a hydrogel.

In certain embodiments, a hydrogel of this invention can comprise thiolated extracellular matrix (ECM) molecules. Such thiolated ECM molecules can include, but are not limited to, thiolated collagen, thiolated gelatin, thiolated laminin, thiolated fibronectin, thiolated heparin, thiolated hyaluronan (HA), any thiol group-containing peptide sequence, or any combination thereof. By using different ratios of these thiolated components and adjusting the cross-link density, a series of hydrogels can be formulated with a range of mechanical properties and customizable biomolecule release profiles. Thus, in some embodiments of the invention, the hydrogel can be a thiolated hyaluronan-collagen-fibronectin hydrogel. In other embodiments, the hydrogel can be a HA-gelatin hydrogel.

In some embodiments of the present invention, the hydrogel comprises one or more (e.g., 1, 2, 3, 4, 5, 6, 7, 8, 9, 10, 11, 12, 13, 14, 15, 16, 17, 18, 19, 20, etc.) biomolecule(s) of this invention. Thus, the biomolecules of the hydrogel include, but are not limited to, a differentiation stimulating biomolecule, a chemotaxis stimulating molecule, a proliferation stimulating biomolecule, a mobilization stimulating biomolecule, or any combination thereof, as described above.

In particular embodiments of the invention, the biomolecule of the hydrogel can be hepatocyte growth factor (HGF). In other embodiments of the invention, the biomolecule of the hydrogel can be a bone morphogenic protein-2 (BMP-2).

It is also contemplated as part of this invention that the hydrogel can be contacted with the scaffold prior to and/or after the scaffold is delivered to the subject. Thus, the hydrogel can be associated with the scaffold prior to and/or post implantation. The hydrogel can be introduced (“loaded”) into the scaffold by immersion or other contact of the scaffold with the hydrogel and/or the hydrogel's pre-gel constituents. The association of the hydrogel with the scaffold can be facilitated further by a physical means such as sonication or centrifugation. The hydrogel can be loaded by single or multiple contact events and/or injections and these contact events can occur pre- and/or post-implantation. The association between the scaffold and hydrogel can be temporary (e.g., no permanent fixation means used, may leak out over a period of time) or the association between the scaffold and hydrogel can be carried out by physically locking the hydrogel into place in the scaffold by hydrogel gelling and/or crosslinking post-loading (e.g., two completely independent but interpenetrating networks or IPNs without covalent linking between the two). The association between the scaffold and hydrogel can also be carried out by locking the hydrogel into place via induction (e.g., heat, etc), in which the hydrogel chemically interacts with the scaffold.

The present invention further provides methods of producing a scaffold comprising photocurable polysaccharide and protein, the method comprising: a) adding a photocurable polysaccharide in a solvent (e.g., DMSO, DMF, DMAC, acetone, dichloromethane and 1, 1, 1, 3, 3, 3-hexafluoro-2-propanol, any other known solvent or any combination thereof) to a protein-solvent mixture to make a polysaccharide-protein-solvent mixture; b) adding a photoinitiator to the mixture of step (a) and c) exposing the polysaccharide-protein-solvent mixture of step (b) to an irradiation source (e.g., ultraviolet (UV) light) to photocure the photocurable polysaccharide, whereby a scaffold comprising photocurable polysaccharide and protein is produced.

In some embodiments of the methods of this invention, the polysaccharide-protein-solvent mixture is allowed to set for a period of time of zero hours to about five days (e.g., one hour, two hours, four hours, eight hours, one day, two days, etc., including any time point between zero hours and five days not specifically recited herein) at a temperature of about 10 degrees Celsius to about 60 degrees Celsius (e.g., in a range from about 20 degrees to about 30 degrees Celsius) prior to exposure to the irradiation source (step (c)). As one example, to produce a scaffold to be used for cartilage regeneration, the setting time of the scaffold is zero hours (i.e., the scaffold is not allowed a setting time before the next step is carried out). As another example, a scaffold to be used for neuron regeneration can have a setting time of about five days. The setting time will regulate the mechanical properties of the scaffold and a scaffold with a longer setting time will be softer than a scaffold with a shorter setting time. With no setting time, the higher strength and better elasticity will be beneficial for cartilage regeneration. For neuron regeneration, the long setting time results in disappearance of large pores and the scaffold becomes softer.

The polysaccharide in the polysaccharide-solvent mixture can be provided at a concentration (in weight/weight/weight, w/w/w) in a range from about 1% (w/w/w) to about 20% (w/w/w). Thus, the polysaccharide in the polysaccharide-solvent mixture can be provided at a concentration in weight percent of about 1%, 2%, 3%, 4%, 5%, 6%, 7%, 8%, 9%, 10%, 11%, 12%, 13%, 14%, 15%, 16%, 17%, 18%, 19%, 20% or any fraction thereof within this range not specifically recited herein (e.g., 7.5%; 12.25%). In embodiments of the invention in which the polysaccharide is photocurable chitosan and the solvent is DMSO, the photocurable chitosan in the chitosan-DMSO mixture can be provided at a concentration (in weight/weight/weight, w/w/w) in a range from about 1% (w/w/w) to about 20% (w/w/w). Thus, the chitosan in the chitosan-DMSO mixture can be provided at a concentration in weight percent of about 1%, 2%, 3%, 4%, 5%, 6%, 7%, 8%, 9%, 10%, 11%, 12%, 13%, 14%, 15%, 16%, 17%, 18%, 19%, 20% or any fraction thereof within this range not specifically recited herein (e.g., 7.5%; 12.25%). In particular embodiments, the chitosan in the chitosan-DMSO mixture can be provided at a concentration in weight percent in a range from about 5% to about 6%, from about 5% to about 6.5%, from about 5% to about 7%, from about 6% to about 7%, from about 6% to about 7.5% or from about 7% to about 7.5, and the like.

The protein in the protein-solvent mixture can be provided at a concentration (in weight/weight/weight, w/w/w) in a range from about 1% (w/w/w) to about 20% (w/w/w). Thus, the protein in the protein-solvent mixture can be provided at a concentration in weight percent of about 1%, 2%, 3%, 4%, 5%, 6%, 7%, 8%, 9%, 10%, 11%, 12%, 13%, 14%, 15%, 16%, 17%, 18%, 19%, 20% or any fraction thereof within this range not specifically recited herein (e.g., 7.5%; 12.25%). In embodiments of the invention in which the protein is gelatin and the solvent is DMSO, the gelatin in the gelatin-DMSO mixture can be provided at a concentration (in weight/weight/weight, w/w/w) in a range from about 1% (w/w/w) to about 20% (w/w/w). Thus, the gelatin in the gelatin-DMSO mixture can be provided at a concentration in weight percent of about 1%, 2%, 3%, 4%, 5%, 6%, 7%, 8%, 9%, 10%, 11%, 12%, 13%, 14%, 15%, 16%, 17%, 18%, 19%, 20% or any fraction thereof within this range not specifically recited herein (e.g., 7.5%; 12.25%). In particular embodiments, the gelatin in the gelatin-DMSO mixture can be provided at a concentration in weight percent in a range from about 5% to about 6%, from about 5% to about 6.5%, from about 5% to about 7%, from about 6% to about 7%, from about 6% to about 7.5% or from about 7% to about 7.5, and the like.

The photoinitiator of this invention can be any photoinitiator now known or later identified. Nonlimiting examples of a photoinitiator of this invention include Irgacure 2959, Irgacure 149, Irgacure 184, Irgacure 369, Irgacure 500, Irgacure 651, Irgacure 784, Irgacure 907, Irgacure 1800, Irgacure 1850, Darocur 1173 and Darocur, including any combination thereof.

In some embodiments, the scaffold of this invention can be treated with a crosslinking and/or catalyzing agent [e.g., 1-ethyl-(3-3-dimethylaminopropyl carbodiimide hydrochloride (EDC); N,N′-dicyclohexylcarbodiimide (DCC); N,N′-diisoproplycarbodiimide (DIC), genipin and any other crosslinking and/or catalyzing agent known in the art for crosslinking proteins, in any combination]. Thus, in certain embodiments of the methods of this invention, the scaffold is contacted with a solution of EDC. Treatment with EDC results in fabrication of an interpenetrated network (IPN) of the elastic scaffold.

In certain embodiments of the invention, the scaffold produced according to the methods described herein can be boiled. The boiling step can be optional and is carried out to wash off excess protein (e.g., gelatin) and to clarify the morphology and distribution of the photocurable polysaccharide in the scaffolds through SEM.

In further embodiments of this invention, one or more biomolecules are associated with the scaffold. Thus, the methods of this invention for producing a scaffold of this invention further comprise the step of associating one or biomolecules with the scaffold. As stated above, the biomolecules, in the form of proteins, peptides and/or nucleic acids, can be incorporated into the scaffold at any step in the fabrication (pre-, during and/or post-fabrication) of the scaffold. Additionally, as noted above, in other embodiments, the biomolecules, in the form of proteins, peptides and/or nucleic acids, can be delivered directly to the subject according to well known methods.

The present invention additionally provides methods of regenerating tissue (e.g., in a subject in need thereof), comprising contacting the subject with a scaffold of the present invention. In some embodiments the scaffold comprises, consists essentially of and/or consists of one or more biomolecules of this invention in any combination. In particular embodiments, the methods of regenerating tissue in the subject are carried out in the absence of cell transplantation that is recognized as part of the tissue regeneration process, either prior to, during or after contacting the subject with the scaffold. Specifically, tissue regeneration procedures known in the art include the transplantation of cells (autologous and/or allogeneic cells) into the subject and such cells facilitate the tissue regeneration process.

The present invention is an unexpected improvement over such procedures, because the composition of the scaffold of this invention provides for the association therewith of one or more biomolecules that serve to attract the subject's own cells to the site where tissue regeneration is needed or desired, thereby obviating the need for transplanting cells (either autologous or allogeneic) into the subject as part of the tissue regeneration process. Thus, in some embodiments, a scaffold of this invention comprises no cells (i.e., it is a cell-free scaffold) and comprises one or more biomolecules of this invention in any combination. By cell-free, it is meant in some embodiments that the scaffold is prepared according to the methods described herein with the proviso that no cells are added to or contacted with the scaffold during preparation and it is this scaffold thus produced with no cells that is contacted with or introduced into a subject of this invention (e.g., a subject in need of tissue regeneration). The cell free scaffold is contacted with or introduced into the subject in the absence of a cell transplant, either prior to, simultaneously with, or after such contact.

As used herein, the terms “cell transplant” or “transplantation of cells” means the introduction from an external source of cells into a recipient. The cells can be the recipient's own cells that had been removed previously (i.e., autologous or homologous transplant) or the cells can be from a donor (i.e., an allogeneic, isologous or heterologous transplantation of cells not from the recipient).

Thus, the present invention provides a method of regenerating tissue in a subject, comprising contacting the subject with a scaffold of this invention, thereby attracting cells already present in the subject under natural conditions (i.e., not previously removed from the subject and returned to the subject as an autologous or homologous transplant) to the site of tissue regeneration and stimulating or activating said cells to regenerate tissue. In some embodiments, the subject may receive a cell transplant that is not a cell transplant that directly facilitates tissue regeneration.

Tissues that can be regenerated using this method include, but are not limited to, any hard or soft tissue, such as cartilage, bone, dental tissue, skeletal muscle, smooth muscle, skin, blood vessel, heart, liver, kidney, pancreas, brain, spinal cord, nerve tissue, etc., as would be well known in the art.

A site of contact for the scaffold of the present invention includes, but is not limited to, inside and/or in proximity to a joint space, a muscle, bone, connective tissue; an organ, a blood vessel, skin, a body cavity, etc., including any combination thereof.

Methods of contacting the subject in need thereof with the scaffold of the present invention include but are not limited to surgical implantation, placement into a body cavity, injection, topical delivery, or any combination thereof.

The term “subject” as used herein includes any subject in which tissue regeneration according to the present invention can be carried out. In some embodiments, the subject can be a mammalian subject (e.g., dog, cat, horse, cow, sheep, goat, monkey, rat, mouse, lagomorphs, ratites etc.), and in particular a human subject (including both male and female subjects, and including neonatal, infant, juvenile, adolescent, adult, and geriatric subjects, further including pregnant subjects). A subject in need thereof includes, but is not limited to, a subject having tissue that is injured, damaged, diseased and/or has an age related disorder and thus, is in need of regeneration.

The present invention further provides delivering nanoparticles and/or microspheres comprising at least one biomolecule to the subject. Nanoparticles and microspheres comprising at least one biomolecule can be used for short-term biomolecule or signal delivery by encapsulating the biomolecule in nanospheres and/or microspheres. Material selection for the fabrication of the nanoparticles and microspheres and sphere diameter determines the length of the delivery period, as is well known in the art. Thus, in some embodiments, the nanoparticles and microspheres can be biodegradable. In other embodiments, the nanoparticles and/or microspheres can be nonbiodegradable. The nanoparticles and/or microspheres of this invention can be produced from any biocompatible material known in the art for such production.

The present invention further provides nanoparticles and/or microspheres comprising at least one biomolecule, wherein the at least one biomolecule is a biomolecule as described above. Accordingly, the biomolecule includes, but is not limited to, a differentiation stimulating biomolecule, a chemotaxis stimulating molecule, a proliferation stimulating biomolecule, a mobilization stimulating biomolecule, or any combination thereof, as described above. Other therapeutic agents or biomolecules that can be provided via the microspheres and nanoparticles include, but are not limited to, PNPX (para-nitrophenyl-beta-D-xyloside), cAMP, prolyl hydroxylase inhibitors (PHIs), and brain-derived neurotrophic factor.

The microspheres of the present invention can be in a size range of about 5 μm to about 50 μm. Thus, the microspheres can be 5 μm, 10 μm, 15 μm, 20 μm, 25 μm, 30 μm, 35 μm, 40 μm, 45 μm, 50 μm, and the like or any combination thereof. In other embodiments, the microspheres can be in a range from about 5 μm to about 10 μm, from about 5 μm to about 15 μm, from about 5 μm to about 20 μm, from about 5 μm to about 25 μm, from about μm to about 30 μm, from about 5 μm to about 35 μm, from about 5 μm to about 40 μm, from about 5 μm to about 45 μm, from about 10 μM to about 15 μm, from about 10 μm to about 20 μm, from about 10 μm to about 25 μm, from about 10 μm to about 30 μm, from about 10 μm to about 35 μm, from about 10 μm to about 40 μm, from about 10 μM to about 45 μm, from about 10 μm to about 50 μm, from about 15 μm to about 20 μm, from about 15 μm to about 25 μm, from about 15 μm to about 30 μm, from about 15 μm to about 35 μm, from about 15 μm to about 40 μm, from about 15 μm to about 45 μm, from about 15 μm to about 50 μm, from about 20 μm to about 25 μm, from about 20 μm to about 30 μm, from about 20 μm to about 35 μm, from about 20 μm to about 40 μm, from about 20 μm to about 45 μm, from about 20 μm to about 50 μm, from about 25 μm to about 30 μm, from about 25 μm to about 35 μm, from about 25 μm to about 40 μm, from about 25 μm to about 45 μm, from about 25 μm to about 50 μm, from about 30 μm to about 35 μm, from about 30 μm to about 40 μm, from about 30 μm to about 45 μm, from about 30 μM to about 50 μm, from about 35 μm to about 40 μm, from about 35 μm to about 45 μm, from about 35 μm to about 50 μm, from about 40 μm to about 45 μm, from about 40 μm to about 50 μm, from about 45 μm to about 50 μm, and the like.

The nanoparticles of the present invention are in a size range of about 20 nm to about 50 nm. Thus, the nanoparticles can be 20 nm, 25 nm, 30 nm, 35 nm, 40 nm, 45 nm, 50 nm, and the like or any combination thereof. In other embodiments, the microspheres can be in a range from about 20 nm to about 25 nm, from about 20 nm to about 30 nm, from about 20 nm to about 35 nm, from about 20 nm to about 40 nm, from about 20 nm to about 45 nm, from about 20 nm to about 50 nm, from about 25 nm to about 30 nm, from about 25 nm to about 35 nm, from about 25 nm to about 40 nm, from about 25 nm to about 45 nm, from about 25 nm to about 50 nm, from about 30 nm to about 35 nm, from about 30 nm to about 40 nm, from about 30 nm to about 45 nm, from about 30 nm to about 50 nm, from about 35 nm to about 40 nm, from about 35 nm to about 45 nm, from about 35 nm to about 50 nm, from about 40 nm to about 45 nm, from about 40 nm to about 50 nm, from about 45 nm to about 50 nm, and the like.

The nanoparticles and/or microspheres of the present invention are delivered to the subject via a variety of methods, including, but not limited to, injection, surgical implantation, delivery into a body cavity, topical application, and any combination thereof. The nanoparticles and/or microspheres of this invention can be present in the scaffold of this invention and are therefore delivered to the subject via contacting of the subject with the scaffold. The nanoparticles and/or microspheres can also be delivered to the subject separately from the scaffold.

In a specific embodiment, the present invention provides a method of regenerating cartilage in a subject having a partial cartilage defect, a full thickness defect and/or an osteochondral defect, the method comprising contacting the defect(s) in the subject with a scaffold of the present invention. The scaffold can comprise one or more differentiation biomolecules. The scaffold can further comprise one or more chemotaxis and proliferation biomolecules.

In some embodiments of the invention, the method of cartilage regeneration provides binding a differentiation biomolecule(s) to a cross-linked polysaccharide-protein scaffold. In other embodiments, the scaffold can additionally comprise one or more chemotaxis and proliferation biomolecules. In particular embodiments, the scaffold is a cross-linked chitosan-gelatin scaffold.

In some embodiments, the method of regenerating cartilage in a subject having a partial cartilage defect, a full thickness defect and/or an osteochondral defect further comprises delivering a nanoparticle and/or microsphere comprising at least one biomolecule to the subject, wherein the delivery is directly into and/or in proximity to a joint space having the defect(s). In other embodiments, the biomolecule associated with the nanoparticle and/or microspheres is a biomolecule that stimulates the mobilization of mesenchymal stem cells. In still other embodiments, the biomolecule is hepatocyte growth factor (HGF). In additional embodiments, the at least one biomolecule is a biomolecule as described above. The nanoparticle and/or microsphere can be delivered to the subject via the scaffold and/or the nanoparticle and/or microsphere can be delivered to the subject separately from the scaffold, either prior to, simultaneously with and/or after contacting the subject with the scaffold.

Embodiments of the present invention further provide a kit comprising one or more of the compositions described herein and optionally instructions for use and/or administration. It would be well understood by one of ordinary skill in the art that the kits of this invention can comprise one or more containers and/or receptacles to hold the reagents of the kit, along with appropriate reagents and directions for using the kit, as would be well known in the art. Each of these components of the kit can be combined in the same container and/or provided in separate containers.

The present invention is more particularly described in the Examples set forth below, which are not intended to be limiting of the embodiments of this invention.

### EXAMPLES

### Example 1

### Scaffold Preparation

Chemically modified photocurable chitosan was synthesized according to the method described previously²³. Briefly, 1 g chitosan was dissolved into methanesulfonic acid while constantly stirring for 25 minutes, followed by dropwise addition of a mixture of 1.1 g benzoyl chloride and 1.227 g methacryloyl chloride. The solution was kept at room temperature with stirring for another 30 minutes before it was added dropwise into an aqueous solution of ammonium hydroxide (100 ml 5 n ammonium hydroxide solution+600 ml DI water). The precipitate was filtered and washed 10 times with DI water to remove the reagent and solvent residues. Finally, the product was dried in vacuum over P₂O₅ for 2 days. The resulting chitosan has a 0.85 degree of deacetylation, a 0.4 graft degree of benzoic groups, and a 0.93 graft degree of methacrylate groups, as determined by ¹H NMR spectroscopy²³.

For gelatin-chitosan (Gtn-Cht) scaffold fabrication, 2 g gelatin was dissolved into 40 g DMSO to reach 5% (w/w) solution under constant stirring. An appropriate amount of chemically modified photocurable chitosan DMSO solution containing 0.5% (wt % based on chitosan) Iragure 2959 was added into the 5% gelatin solution under stirring to obtain a 5% gelatin-5% photocurable chitosan-DMSO mixture, and a 5% gelatin-7.5% photocurable chitosan-DMSO mixture, respectively. The mixture was then slowly poured into molds of circular disc morphology with 2 mm in depth and 8 mm in diameter, and set for different lengths of time (0, 8, 12, and 16 hours for 5% gelatin-5% chitosan-DMSO mixture, and 0, 8, 24, 48, and 56 hours for 5% gelatin-7.5% chitosan-DMSO mixture, respectively) prior to exposure to UV light for 2 minutes to crosslink the chitosan. The photocured discs were immersed into deionized (DI) water for 24 hours and washed several times to remove solvent residues and then freeze dried. A set of the discs underwent boiling water treatment to remove gelatin. Prior to immersion into DI water, another set of the photocured discs was further crosslinked by immersion into a 4:1 acetone-water (v/v) solution containing 1% (w/v) 1-ethyl-(3-3-dimethylaminopropyl carbodiimide hydrochloride) (EDC) at 4° C. overnight, which crosslinked gelatin, and gelatin with chitosan. The discs were then washed and hydrated with DI water. The hydrated discs were frozen at −20° C., and lyophilized. The morphologies of the discs were examined by SEM.

Scaffold characterizations: Rheological measurements were performed on the hydrated discs using an AR-G2 model stress controlled rheometer (T.A. Instruments, U.K.) with 8 mm parallel plate geometry at 25° C. By adjusting the upper plate, a 10% compressive strain was applied to all the discs. Frequency sweep experiments were carried out at 0.1% shear strain between 0.01 and 100 Hz (i.e., 0.0628 and 62.8 rad/s). The discs made from 5% and 7.5% chitosan without gelatin were also measured as controls.

The compressive properties of the scaffolds were investigated using the Dynamic Mechanical Analyzer Q800 (DMAQ800) and the compression test was carried out with a constant strain rate at 1 mm/min and trigger force of 18 N. The initial elastic modulus was calculated based upon the slope of the stress-strain curve at low strains (<3%). For the cyclic compression test, under a constant strain rate of 1 mm/min, the scaffolds were first compressed to 60% strain and then recovered to 30% strain by retreating the force. After that, the scaffolds were repeatedly compressed and recovered between 30% and 60% strain at 1 mm/min strain rate for 4 more cycles. Each measurement was performed three times and averaged.

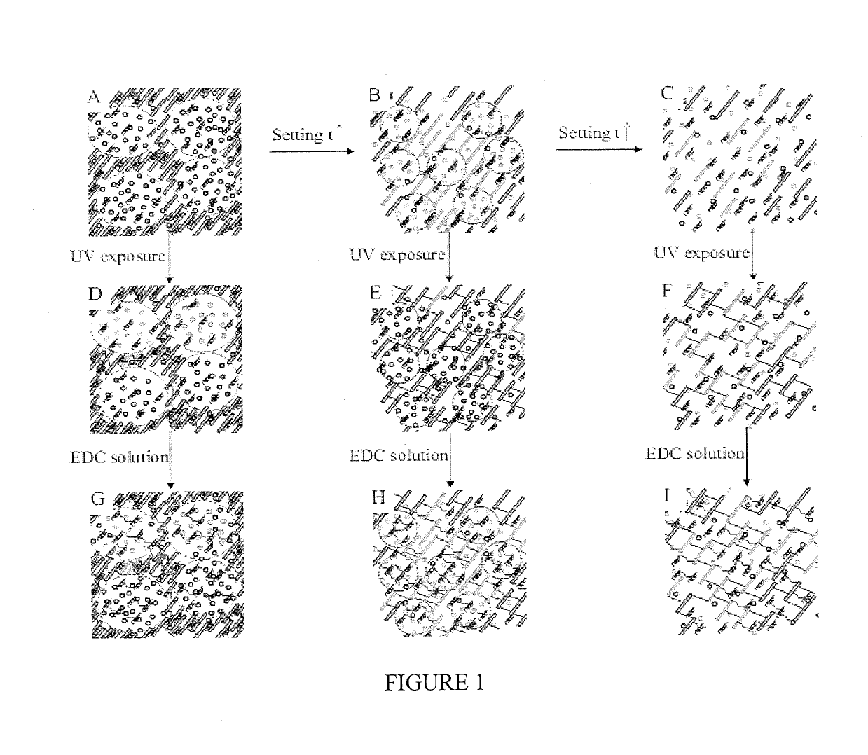

In vitro Cell Culture: For in vitro cell culture, primary bovine osteoblasts were cultured in a 75 mm² flask until confluence. The cells were harvested and counted. A cell suspension containing 80,000 bovine osteoblasts was seeded per re-hydrated disc that was sterilized by ETO gas. Two hours after the cell seeding, culture medium was added into the culture plate. The cell-disc compound was fixed with 4% paraformaldehyde for 30 minutes after 48 hours of culture in incubator (37° C., 5% CO₂). Actin was stained in green using Alexa Fluor 488 conjugated phalloidin and the nuclei were stained using Draq-5. The stained samples were observed and imaged using a Leica laser confocal microscope.
Results. FIG. 1 shows a proposed scheme of interactions between chitosan and gelatin in DMSO. In this scheme, the setting time is the time prior to the crosslinking of either one of the two components. Setting allows free chain motility/configuration changes, and the interactions of individual molecules of chitosan and gelatin. Within the setting time, transient phase separation occurs where gelatin interacts strongly with chitosan via electrostatic interactions to form a biopolymer-rich phase (soluble and insoluble complex coacervate), while gelatin interacts weakly with DMSO molecules via hydrogen-bonding, which forms a solvent-rich phase. Meanwhile, DMSO molecules are able to infiltrate into the biopolymer-rich phase over time via diffusion (entropy-gaining) and non-specific interactions (e.g., hydrogen bonding, hydrophobic interactions). As a result, the solvent-rich phase co-exists with the biopolymer-rich phase either as interstitial bulk or as bound to the strongly interacting biopolymer chains. Setting of the chitosan-gelatin-DMSO system is terminated by crosslinking one of the two components, for instance, chitosan, upon UV exposure.

Crosslinking of chitosan via UV light transforms its structure into an interconnected network, thus inhibiting the chain motility and interactions of individual chitosan molecules with gelatin, and fixing in place the conformation of chitosan in the coacervate (FIGS. 1D, 1E, 1F). Macroscopically, the system undergoes phase transition from liquid to solid after crosslinking of chitosans through photo-curing. Further crosslinking of gelatin in 1-ethyl-(3-3-dimethylaminopropyl carbodiimide hydrochloride) (EDC or EDAC) solution preserves the overall morphology of the gelatin-rich phase through stabilizing the conformation of gelatin in the coacervate (FIGS. 1G, 1H, 1I). EDC can catalyze the reaction between amine and carboxyl groups, thus crosslinking gelatin molecules and/or gelatin molecules with chitosan chains¹⁷. Under each condition, extension of the setting time allows better infiltration and dispersion of DMSO molecules within the biopolymer-rich phase and therefore reduces phase separation in the system (FIGS. 1A, 1B, 1C). Beyond a certain point of the setting time, complete dispersion of solvent in the biopolymers occurs, resulting in phase homogeneity in the system, and the complete disappearance of the solvent-rich phase in the gelatin-chitosan coacervate (FIGS. 1C, 1F, 1I). According to the proposed scheme (FIG. 1), freeze drying of the system after curing would produce a 3-D chitosan-gelatin scaffold containing microporous structures that are left by the solvent-rich phase in the coacervate (the dotted circles in FIG. 1).

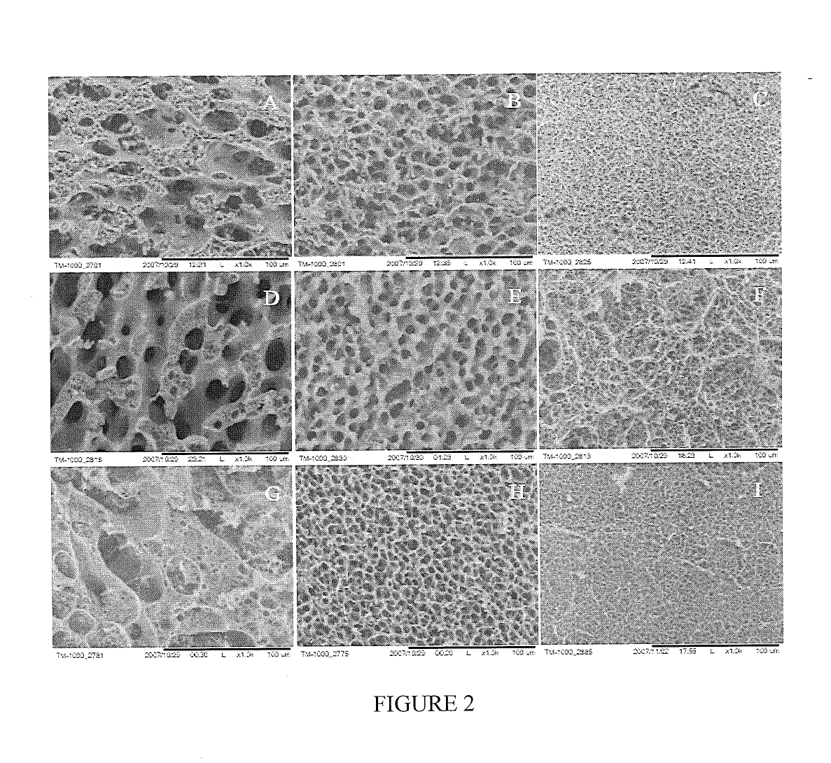

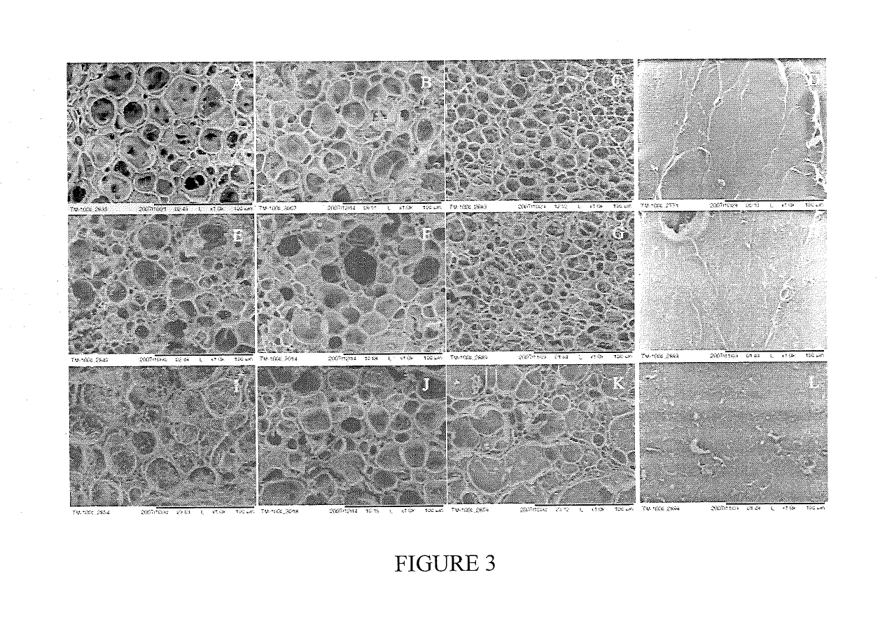

To harness the chitosan-gelatin interactions in a DMSO solution for the creation of hybrid porous scaffolds with unique physical and morphological properties, the pore formation dynamics in the chitosan-gelatin coacervate were examined as a function of the interaction parameters, such as the setting time, the ratio of gelatin to chitosan, and with or without the crosslinking of gelatin with EDC. Two sets of experiments were performed using different ratios of gelatin (Gtn) to chitosan (Cht): 5% Gtn-5% Cht-DMSO, and 5% Gtn-7.5% Cht-DMSO, respectively. All the concentrations refer to weight percentages in the solution. In each set of experiments, the systems that underwent different treatments and different setting times for scaffold production were freeze-dried and SEM examination was performed (FIG. 2 and FIG. 3).

The boiling water immersion/washing of the freeze-dried scaffolds was performed to remove the uncrosslinked gelatin by dissolving it, leaving the crosslinked chitosan skeleton intact in the coacervate (FIGS. 2D, 2E, and 2F). Comparison of the scaffolds obtained in the beginning of the setting, but with different treatments, indicates differences in the pore sizes and morphologies. With 5% Gtn-5% Cht-DMSO system, large pores of an average of 30-40 μm in diameter (FIGS. 2A and 2D) were seen in the scaffolds, implying the separation of the solvent-rich phase, which creates the pores, and the biopolymer-rich phase, which forms the scaffold skeleton in the system. In fact, upon the addition of chitosan into the gelatin-DMSO solution, the system became a little cloudy, perhaps due to the formation of microscopic coacervate droplets of chitosan and gelatin. Strong electrostatic interactions between chitosan and gelatin in DMSO may initiate intermolecular insoluble aggregate formation comprised of charge neutralized chitosan-gelatin complexes⁶. However, the insoluble aggregates of chitosan-gelatin complexes did not undergo precipitation, instead, over time they disappeared and the solution became clear (10 hours for 5% Gtn-5% Cht-DMSO system, and 36 hours for 5% Gtn-7.5% Cht-DMSO system).

The differences in pore sizes and morphologies of the scaffolds before (FIG. 2A) and after the boiling water removal of gelatin (FIG. 2D) suggest a biphasic distribution of gelatin in both the biopolymer-rich phase and the solvent-rich phase. Gelatin and chitosan were not completely associated and co-localized in the coacervate. While chitosan primarily existed in the biopolymer-rich phase and served as the structural skeleton of the scaffolds (FIG. 2D), gelatin was present in both phases as masking and filling materials in the scaffolds (biopolymer-rich phase) and the pores (solvent-rich phase) (FIG. 2G). Following the boiling water treatment, the pores exhibited more clear morphologies with discernible edges, along with the disappearances of the filling structures inside the pores, again suggesting the biphasic presence of gelatin in both the biopolymer-rich phase and the solvent-rich phase (FIGS. 2A, 2D, 2G). Comparisons of the scaffold pore morphologies under the same set of treatments (boiling water vs. EDC crosslinking), but with prolonged setting time (8 hours), indicate the same trend (FIGS. 2B, 2E, 2H). Extension of the setting time may allow better infiltration, integration, and dispersion of DMSO molecules (the solvent-rich phase) into the intramolecular and intermolecular space within the chitosan-gelatin complex coacervate. Therefore, incremental reduction in the pore sizes was documented over the setting time (8 and 12 hours), indicating reduction in the sizes of the solvent-rich phase in the biopolymer complex. Beyond a critical point of setting time (12 hours), DMSO molecules completely dispersed into the complex, leading to phase homogeneity of the system, and the disappearance of macropores (solvent-rich phase) in the scaffolds.

A parallel set of experiments using a different ratio of gelatin to chitosan (5% Gtn-7.5% Cht-DMSO system) indicated a similar trend of changes in the scaffold pore sizes and morphologies as a function of setting time (FIG. 3). Compared to the 5% Gtn-5% Cht-DMSO system, more chitosan molecules were available to interact with gelatin, forming a complex coacervate containing chitosan skeleton with denser structures (FIGS. 3A, 3E, 3I vs. FIGS. 2A, 2D, 2G). Pores of smaller sizes were embedded in the complex, and oftentimes these were surrounded and filled by gelatin (FIG. 3I). Due to the increased density of the complex coacervate, it took longer for DMSO molecules to penetrate and disperse into the coacervate. Therefore, there were not many differences in the pore sizes and morphologies of the scaffolds at 0, and 8 hours setting time points (FIGS. 3A, 3E, 3I vs. FIGS. 3B, 3F, 3J), even though the differences in the scaffolds were pronounced at these two setting time points with the 5% Gtn-5% Cht-DMSO system (FIGS. 2A, 2D, 2G vs. FIGS. 2B, 2E, 2H). Instead of 12 hours as with the 5% Gtn-5% Cht-DMSO system, it took 48 hours for DMSO to completely disperse into the biopolymer complex, and the macropores (solvent-rich phase) in the scaffolds to disappear (FIGS. 3D, 3H, 3L). For the 5% Gtn-7.5% Cht-DMSO system, the temporal dynamics of pore size reduction were also different from that of the 5% Gtn-5% Cht-DMSO system (FIG. 3 vs. FIG. 2). Again, morphological comparison of the scaffolds before and after boiling water removal of gelatin indicates a biphasic distribution of gelatin both in the biopolymer-rich complex and solvent-rich phase, preferably as masking and filling materials (FIG. 3A, 3B, 3C vs. 3E, 3F, 3G). The results of the average pore size and porosity measurements of the scaffolds produced under different conditions are listed in Table 1 and Table 2.

TABLE 1
The average pore sizes of the scaffolds made under different conditions (μm).

| | 5% G | 5% G | 5% G | 5% G | 5% G | 5% G | 5% G |
|---|---|---|---|---|---|---|---|
| | 5% C | 5% C | 5% C | 7.5% C | 7.5% C | 7.5% C | 7.5% C |
| | 0 hr | 8 hrs | 12 hrs | 0 hrs | 8 hrs | 24 hrs | 48 hrs |
| UV | 31.87 ± 0.24 | 9.40 ± 0.13 | 1.78 ± 0.05 | 22.33 ± 0.15 | 18.58 ± 0.14 | 12.19 ± 0.09 | 0 |
| Exposure | | | | | | | |
| Boiled | 35.62 ± 0.26 | 13.24 ± 0.12 | 3.78 ± 0.06 | 11.27 ± 0.11 | 16.06 ± 0.15 | 11.01 ± 0.04 | 0 |
| EDC | 27.55 ± 0.19 | 8.23 ± 0.11 | 1.43 ± 0.07 | 29.53 ± 0.14 | 23.375 ± 0.17 | 14.37 ± 0.10 | 0 |
| solution | | | | | | | |

TABLE 2
The porosity of the scaffolds made under different conditions.

| | 5% G | 5% G | 5% G | 5% G | 5% G | 5% G | 5% G |
|---|---|---|---|---|---|---|---|
| | 5% C | 5% C | 5% C | 7.5% C | 7.5% C | 7.5% C | 7.5% C |
| | 0 hr | 8 hrs | 12 hrs | 0 hrs | 8 hrs | 24 hrs | 48 hrs |
| UV | 80% ± 1% | 74% ± 5% | 47% ± 7% | 77% ± 4% | 79% ± 5% | 69% ± 3% | 0 |
| Exposure | | | | | | | |
| Boiled | 85% ± 1% | 77% ± 4% | 60% ± 6% | 79% ± 1% | 80% ± 2% | 72% ± 4% | 0 |
| EDC | 67% ± 2% | 72 ± 3% | 40% ± 3% | 60% ± 2% | 63% ± 5% | 54% ± 3% | 0 |
| solution | | | | | | | |

The observations of the changes in the pore sizes and morphologies in the hybrid scaffolds as a function of interaction parameters (e.g., the setting time, the ratio of gelatin to chitosan, and the crosslinking of gelatin) are consistent with the scheme of interactions between gelatin and chitosan in an organic solvent (DMSO) solution as set forth in FIG. 1. The phase separation initially observed in coacervation between a biopolymer-rich phase and a solvent-rich phase may be driven by the electrostatic and biopolymer-solvent interactions. Prolonged setting time allows the infiltration and better dispersion of the solvent-rich phase in the biopolymer-rich phase, a process mediated by solvent-biopolymer interactions and also associated with net entropy gain.

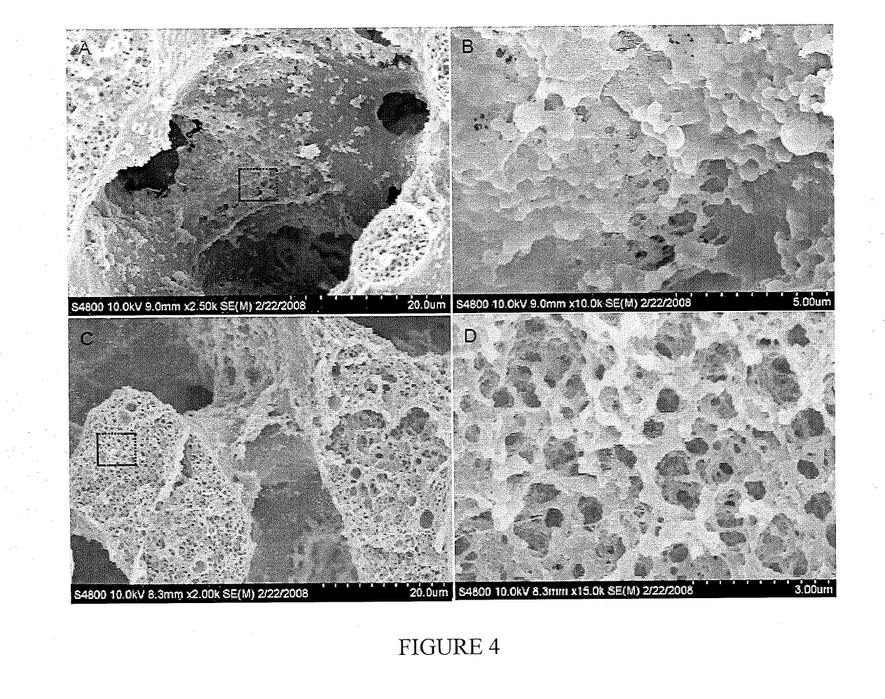

Close examination of the surfaces and inner structures of the hybrid chitosan-gelatin scaffolds by SEM at high magnifications revealed the presence of nanoscale structures (e.g., pores, beads) on the inside and surface of the scaffold skeleton (FIG. 4). Because the surfaces of the scaffold skeleton are the interfaces between the two phases (biopolymer-rich phase and solvent-rich phase) during the coacervation process, these observations again suggest biphasic distribution of gelatin and the ability of gelatin to form nanoscale structures at the interfaces during the transient phase separation. Previously, Ma and colleagues have produced 3-D porous nano-fibrous scaffolds with the walls of the macro-pores covered with nanoscale polymeric fibers¹⁸. Cell culture experiments with osteoblasts using these scaffolds in comparison to solid-walled 3-D porous scaffolds indicated that nanoscale structures at the surfaces were important to the mediation of cell attachment, differentiation, and biomineralization potentially through selectively enhancing the adsorption of specific types of proteins that are favorable for cell-cell interactions, matrix productions, cell-matrix interactions, and bioactivity. In a similar manner, the nanoscale architectures that are created on the skeleton of the hybrid chitosan-gelatin scaffold of the present invention can serve as adhesive domains to promote cell attachment, spreading, ECM production, and functioning. When compared to the existing reports of production of nano-architectured materials¹⁹⁻²¹, different forms of nanoscale architectures on the walls of the 3-D porous scaffolds of the present invention produced by governing a transient phase separation process in a polysaccharide-protein-organic solvent system represents the simplest, easily-controllable method that allows the design of the wall structures of the pores.

To determine whether different types of interactions, as characterized by different binding strength and energy levels, are involved in the association of gelatin with other molecules in the system (chitosan, DMSO), further experiments were performed by subjecting the scaffolds of crosslinked chitosan but uncrosslinked gelatin to a series of treatments with hot water with incrementally increasing temperatures. Because gelatin is readily soluble in hot water, increases in the water temperature will break up the interactions/bonding of gelatin with other molecules in the system. As a result, an increasing amount of uncrosslinked gelatin is eluted from the scaffolds as a function of increasing water temperature. By measuring the amount of gelatin eluted from the scaffolds in comparison to the total amount of gelatin in the starting material, a positive correlation was found between the water temperature and the amount of eluted gelatin. However, even with boiling water, only a small percentage of the gelatin was eluted from the scaffolds, indicating that the energy provided by boiling water was not sufficient to liberate the gelatin molecules that were involved in interactions at high energy levels. These results strongly suggest the presence of different types of interactions between gelatin and chitosan at a multitude of energy levels.

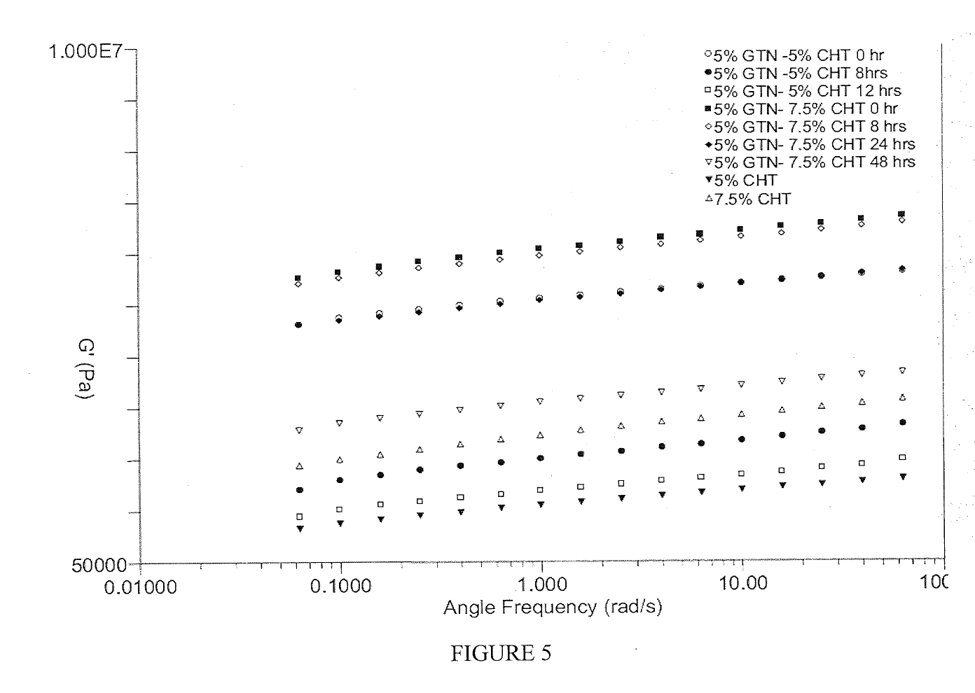

The rheological characteristics/viscoelastic properties of the chitosan-gelatin hybrid scaffolds were evaluated by measuring the storage modulus (G′) and the loss modulus (G″). A storage modulus was used as an index of the elastic component of the material, while a loss modulus was used as a measure of the viscous component. The mechanical properties of natural polymers, such as chitosan, are usually weak. Interactions of natural polymers with proteins or other polymers in solution, whether hydrophobic, electrostatic, or hydrogen bonding in nature, may reinforce the mechanical properties of the scaffolds or complexes obtained from such mixtures⁶. All the scaffolds of the present invention exhibited elastic-dominant characteristics (G′>>G″) and a frequency dependence of G′ across the range tested (FIG. 5).

When compared to the data for pure chitosan scaffolds, gelatin complexation with chitosan increased G′, and therefore reinforced the gelatin-chitosan scaffolds. The range of elasticity of the scaffolds exceeded what has been achieved in any other natural biopolymers with the exception of elastin, demonstrating the ability of the hybrid scaffolds of the present invention to expand the mechanical properties of natural biopolymers. G′ shows a decreasing trend over the setting time regardless of the gelatin-chitosan ratio in the scaffolds (FIG. 5). Because the pore sizes were reduced in the scaffolds as a function of setting time, these observations suggest a role for the macropores formed by the solvent-rich phase in the coacervate in energy-dissipation and buffering to improve the resistance of the scaffolds to deformation. The dynamics of G′ reduction over the setting time in the scaffolds of different gelatin-chitosan ratios was also consistent with the respective temporal dynamics of the changes in the pore sizes in these scaffolds. For example, the 5% Gtn-7.5% Cht scaffolds, which have denser structures and smaller pores, display much higher mechanical strength and a retarded reduction in G′ over the setting time, when compared to that of the 5% Gtn-5% Cht scaffolds, which exhibit sharp drops in G′ over the setting time. This may be due to the fact that higher chitosan content in the coacervate results in denser structures of the 5% Gtn-7.5% Cht scaffolds, which retards the reduction of pore sizes caused by the infiltration of solvent molecules into the coacervate. In addition, for the 5% Gtn-7.5% Cht scaffolds, no difference in G′ was observed at 0 and 8-hour setting times, which is in agreement with the lack of difference in the scaffold pore sizes and morphologies at these two time points.

Natural tissue in the body resides in a complicated biomechanical environment that is constantly subjected to static as well as cyclic mechanical loadings. In evaluating candidate scaffolds for tissue engineering applications, the biomechanical functions in response to static and cyclic mechanical loadings are very important. To this end, the biomechanical behaviors of the scaffolds of the present invention have been evaluated under static vs. cyclic compressive conditions. Three groups of scaffolds were tested: the photocured chitosan-only scaffolds, the photocured Gtn-Cht scaffolds, and the EDC post-cured Gtn-Cht scaffolds. Salt crystals were added to the chitosan-DMSO solution during the scaffold fabrication to achieve the same macro-pores in all the three groups of scaffolds. Again, post-cure of the hybrid scaffolds using EDC solution crosslinked gelatin, and gelatin with chitosan, leading to the formation of an interpenetrating network (IPN) in the scaffolds. Static compression was applied to the scaffolds up to 90% strain. When compared to the photocured chitosan-only scaffolds, the Gtn-Cht scaffolds (the photocured and the EDC post-cured) exhibited much higher compressive strength, as evidenced by greater compressive elastic modulus (Table 3).

TABLE 3
The initial elastic modulus of porous scaffolds

| | Control | Chitosan- | |
|---|---|---|---|
| | (photocured | gelatin | Chitosan-gelatin |
| | chitosan-only) | (photocured) | (EDC post-cured) |
| Initial Elastic | 0.3307 ± 0.0216 | 0.3853 ± 0.0094 | 0.4044 ± 0.0132 |
| modulus | | | |
| (Eᵢₙᵢₜᵢₐₗ, kPa) | | | |

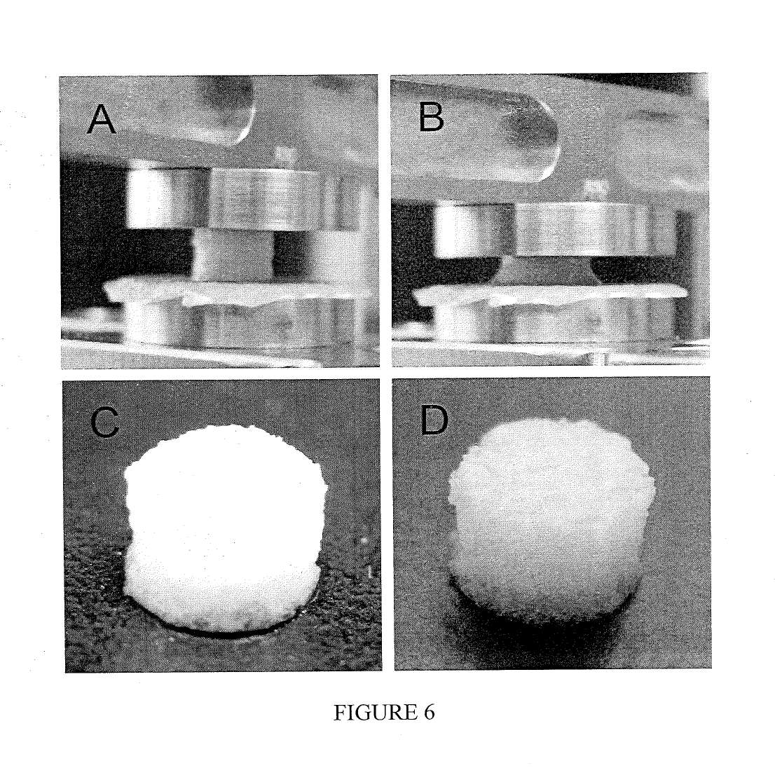

The higher compressive strength of the Gtn-Cht scaffolds may be related to the presence of IPN in the EDC post-cured hybrid scaffolds, which is able to buffer the energy of crushing compressive loading and enhance the resistance of the scaffolds to deformation. Grossly, given sufficient time post-compression, the Gtn-Cht scaffolds were fully recovered to their original dimensions after static compression test up to 90% strain (FIG. 6), whereas the chitosan-only scaffolds failed to recover under the same conditions.

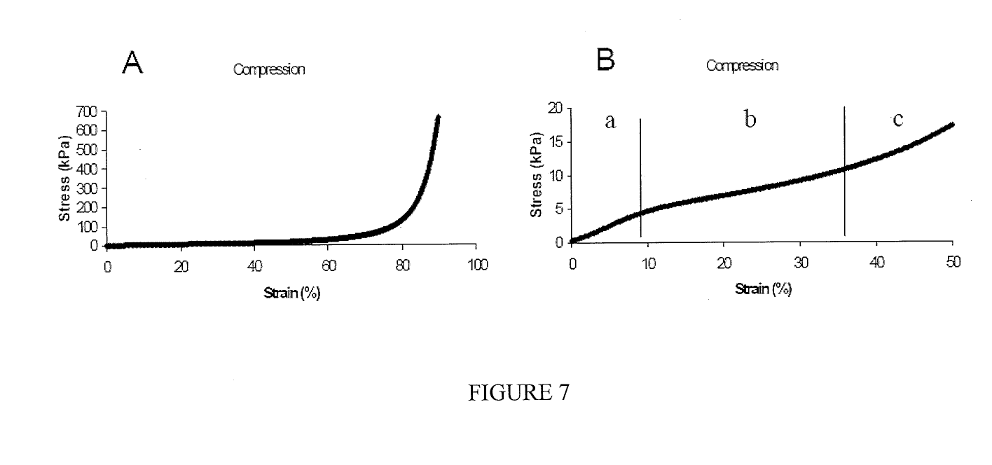

The stress-strain curve of the Gtn-Cht scaffolds at a constant strain rate of 1 mm/min assumes a “J” shape, which is characteristic of foam materials (FIG. 7A)²² Amplification of the curve at low strains (0-50%) (FIG. 7B) indicates that the scaffolds undergo three phases of deformation as a function of strain: linear elasticity (bending) within 10% of strain (FIG. 7B, region a); a plateau (elastic bucking) over medium strains (10% to 35%) (FIG. 7B, region b), and densification at high strains (beyond 35%) (FIG. 7B, region c). The compressive stress-strain behavior of the Gtn-Cht scaffolds conforms to that of an elastic foam²². At low relative strains (FIG. 7B, region a), the macro-pores in the scaffold absorbed the majority of the compressive energy and converted it into a linear elastic deformation of the material primarily by bending of the walls of the macro-pores. At medium strains (FIG. 7B, region b), the wall structure of the macro-pores collapsed and filled into the inner-porous space, giving rise to a plateau (elastic bucking) in the stress-strain curve where the strain continued to increase at a relatively constant level of stress. Eventually, at high strains (FIG. 7B, region c), the inner-porous space was fully filled by the collapsed wall structures and the scaffold started to exhibit a dense solid-like (densification) stress-strain behavior, as evidenced by a rapid increase in the stress necessary to generate small increase in the strain. In comparison, chitosan-only scaffolds collapsed at the plateau region of the stress-strain curve, suggesting the insufficiency in their elastic properties and stiffness to buffer the energy or resist the impact by the compressive loadings.

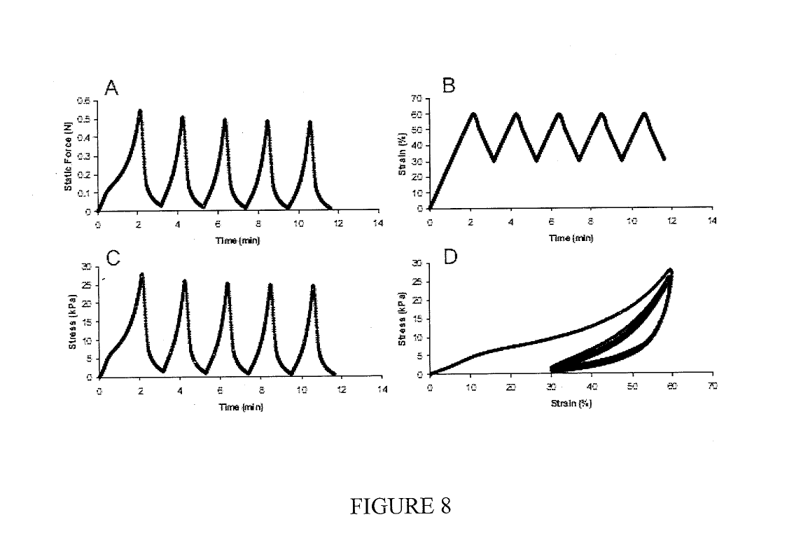

Due to the failure of the chitosan scaffolds during the static compressive test, a cyclic compression test was performed on the Gtn-Cht scaffolds to assess the dynamic load-bearing capacity of the scaffolds at a constant strain rate of 1 mm/min and at strains ranging from 30% to 60% (FIG. 8). At a medium-range strain rate of 1 mm/min, the repeated static force-time or stress-time cycles indicate full recovery of the scaffold in gross dimensions after each cycle within the range of the strain (30% to 60%) (FIG. 8A, 8C). The stress-strain curve (FIG. 8D) indicates that the scaffold recovers to 30% strain instead of the initial no strain condition after the first cycle and reproducibly deforms and recovers along the same stress-strain loop in the following cycles, suggesting a temporal retardation during the recovery process possibly due to the creeping behavior of the scaffold. The difference in the compression and the recovery stress-strain curves also indicates an energy loss during the cyclic compression process; the compressive loading energy absorbed by the scaffold during the compression process was stored in the material and was only partially released during the recovery potentially due to the internalization/dissipation of energy by the material. The area under the stress-strain curve is usually defined as toughness, an index of the material's ability to absorb energy during deformation. Taken together, the static and cyclic compression tests demonstrate that the Gtn-Cht scaffolds are primarily elastic in nature and possess the appropriate strength and toughness to withstand both static and cyclic compressive loadings that simulate physiological conditions.

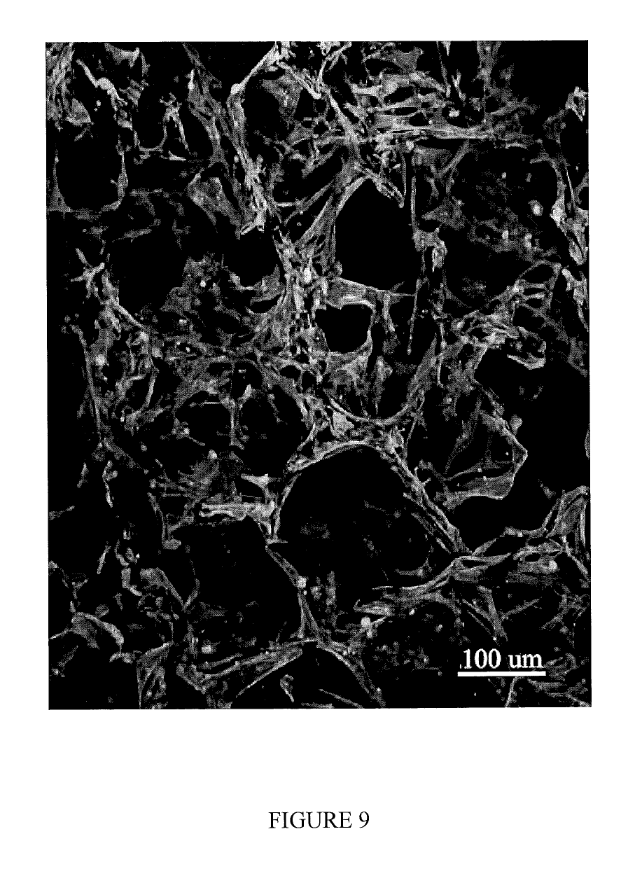

Finally, the potential of the gelatin-chitosan hybrid scaffolds for supporting osteoblast attachment and 3-D organization was evaluated. Osteoblasts that were seeded to the hybrid scaffolds in culture were seen to rapidly attach, spread, and infiltrate into the bulk of the Gtn-Cht scaffolds through the interconnected pores (FIG. 9). The cells exhibited high viability, and were actively proliferating on the scaffolds, indicating a high cell affinity for the gelatin-chitosan hybrid scaffolds that are fabricated by governing the transient phase separation in a polysaccharide-protein-organic solvent system.

Thus, the hybrid scaffolds of the present invention exhibit superior elastic properties, compressive strength and toughness when compared to pure chitosan scaffolds and biopolymer scaffolds reported in the literature. The scaffolds of the present invention were also demonstrated to promote osteoblast attachment, spreading, proliferation, and 3-D organization in vitro.

### Example 2

### Synovial MSC Isolation, Culture, and Characterization

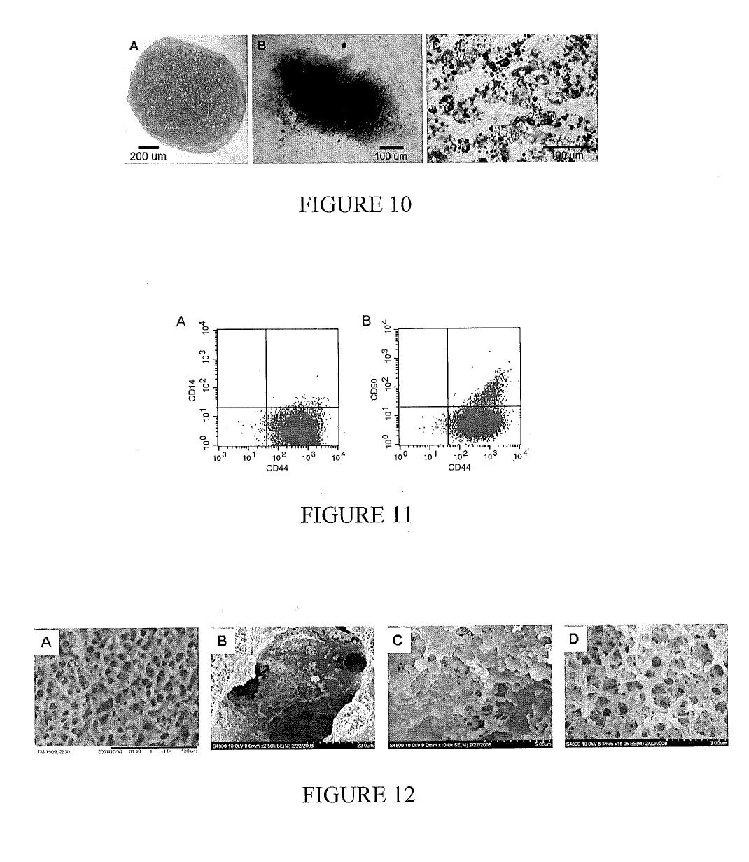

Synovial mesenchymal cells (SMSCs) were isolated from synovial membrane in the rabbit knee. Multipotency of the expanded synovial cells was assessed using standard in vitro differentiation assays for chondrogenesis, osteogenesis, and adipogenesis⁵⁴ (FIG. 10). Limiting dilution assays were used to estimate a colony forming unit efficiency range of 1:13 to 1:52 and an alkaline phosphatase expression range of 1:26 to 1:413. The cell surface antigen profile of passage 2 cultures was investigated with a preliminary panel of monoclonal antibodies to CD14 (macrophage marker), CD44 (hyaluronin receptor), and CD90 (Thy-1) (SeroTech) (FIG. 11). Cell viability was 70.1% (FacsCalibur flow cytometer, CellQuest Pro software both Becton Dickinson). The cells were positive for CD44 and CD90 and negative for CD14. These markers are part of a more extensive panel for MSC antigen expression where CD44 and CD90 are considered important positives⁵⁴. Additional surface markers, including, but not limited to, CD166, CD49a, and Stro-1, can be included to assess differentiation status.

### Example 3

### Cross-Linked Chitosan-Gelatin Elastic Scaffold with Biomolecule Delivery

Highly elastic scaffolds from the natural polymers, chitosan and gelatin, were prepared as described above. Both the chitosan and gelatin were chemically modified to enable polymerization when exposed to light. Because chitosan is positively charged and gelatin is a polyampholyte with both negatively charged and positively charged patches, chitosan and gelatin interact electrostatically, leading to a transition from segregative phase separation to the mixing state. This transition enables controlled formation of a 3D porous structure without using porogens. By varying the chitosan-to-gelatin ratio, setting time, and gelatin crosslinking, 3D porous hybrid scaffolds can be achieved with tunable microstructures across the nano, micro, and macro length scales (FIG. 12). The scaffolds exhibit superior elasticity that has not been previously achieved in any other natural biopolymers except elastin. In vitro culture of the scaffolds with chondrocytes demonstrated cell attachment, spreading, proliferation, and 3D organization.

Heparin was covalently bound to the photo-cured chitosan-gelatin scaffolds to test long-term growth factor release. Scaffolds were incubated in an activated heparin solution for 4 hr and with the growth factor for 12 hr, both at 37° C. A number of growth factors were tested. For example, in vitro release of recombinant human BMP-2 from scaffolds was measured during a 1-month incubation in 0.05% bovine serum albumin in PBS at 37° C. About 6% of the BMP-2 was released after one month, indicating that heparinized scaffolds can deliver growth factors for many months. Four weeks after subcutaneous implantation of the BMP-2-loaded scaffolds, bone formation was observed, indicating that BMP-2 bioactivity is retained with heparin binding.

### Example 6

### Extracellular Matrix Based Hydrogels for Biomolecule Delivery

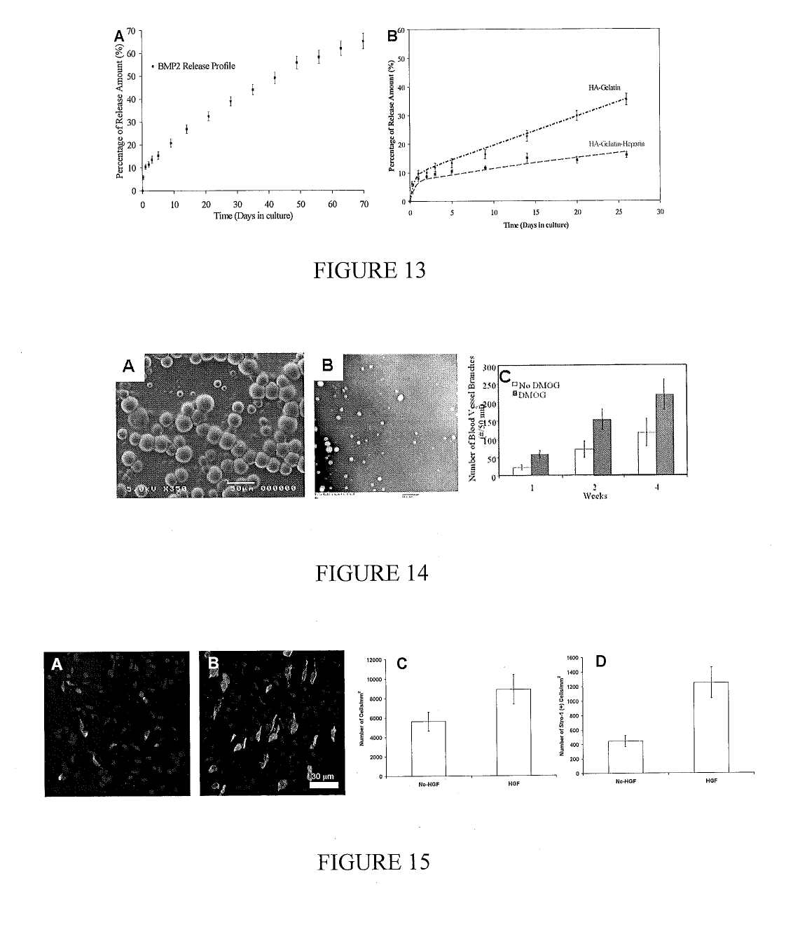

Thiolated ECM molecules, including thiolated collagen, gelatin, laminin, fibronectin, heparin, and hyaluronan (HA), have been used to form hydrogels for long term delivery of biomolecules both in vitro and in vivo. By using different ratios of these thiolated components and adjusting the cross-link density, a series of hydrogels can be formulated with a range of mechanical properties and customizable biomolecule release profiles. For example, a thiolated HA-collagen-fibronectin hydrogel was used to release BMP-2 over a 10 week period in vitro¹⁸ (FIG. 13A). The hydrogel showed a steady release of BMP-2 over the week period.

The effect of immobilized heparin on the controlled release of HGF from HA-gelatin hydrogels in vitro was measured (FIG. 13B). Covalently cross-linked heparin significantly prolonged HGF release. Over the 26-day evaluation period, gels without heparin released a total of 35% of the initially loaded HGF while gels with heparin released only 18% of the HGF. Thus, this demonstrates that the temporal release profile of the bioactive molecules for recruitment and proliferation of SMSC can be tuned by selecting from a series of hydrogels.

### Example 7

### Fabrication of Degradable Microspheres or Nanoparticles Loaded with Biomolecules

A water-in-oil method with combined vigorous sonication and low temperature slow emulsion is used to produce degradable microspheres and nanoparticles loaded with therapeutic agents.⁵⁵⁻⁵⁷ Therapeutic agents that have been loaded onto the degradable microspheres and nanoparticles include PNPX, cAMP, prolyl hydroxylase inhibitors (PHIs), and brain-derived neurotrophic factor. Six weeks of steady release can be achieved with both microspheres and nanoparticles. PHI loaded particles were examined using high-resolution scanning electron microscopy (SEM, Hitachi, Japan). The microspheres had a size range of 5-50 μm; the nanoparticles had a size range of 20-50 nm (FIG. 14A and FIG. 14B, respectively). To evaluate PHI release kinetics, the nanoparticles were placed in a biological buffer and the dimethyloxaloylglycine (DMOG) release was measured for 3 weeks. The release of DMOG displayed zero order kinetics during the 3-week evaluation period. DMOG loaded nanoparticles were placed in a hollow fiber membrane and implanted into rat brain tissue for 1, 2, and 4 weeks. More vascular formation was found in the DMOG group relative to the blank nanoparticle group (FIG. 5C). The degradable nanoparticles with encapsulated biomolecules are useful for mobilization of MSC from rabbit knee synovial membrane.

### Example 8

### Delivering Hepatocyte Growth Factor to Attract Stein Cells

BMSC constitutively express hepatocyte growth factor (HGF), which is an autocrine stimulator of MSC both in animals and humans.⁵⁸⁻⁶¹ HGF has also been shown to be a strong chemotactic factor for MSC mobilization and migration.⁴⁷ A mouse model was used to evaluate the in vivo MSC recruitment potential of HGF-releasing ECM-based hydrogels. Hydrogels with and without HGF were implanted subcutaneously on the back of the mice. After 1 week, the hydrogels were immuno-stained and imaged with a confocal laser microscope (FIGS. 15A, 15B). Cells were shown to have migrated into the scaffolds after 1-week subcutaneous implantation. The number of cells infiltrated into the hydrogels was quantified (FIGS. 15C, 15D). Significantly more cells had infiltrated the HGF releasing hydrogels than the empty hydrogels. An MSC marker, Stro-1, was used to identify non-hematopoietic stromal stem cells.⁶² Significantly more of the cells in hydrogels with HGF expressed Stro-1 (14.2±3.6%) when compared to hydrogels without HGF (7.9±1.3%). This experiment demonstrates that hydrogel scaffolds loaded with HGF may selectively recruit stem cells to the local implantation site. Accordingly, this also demonstrates that HGF, IGF-I, and other factors can be used to selectively recruit stem cells to the scaffold present; e.g., at the site of the tissue defect.

### Example 9

### Screening of Candidate Biomolecules for Mobilization, Chemotaxis, Proliferation, and Differentiation Responses and Determination of the Temporal Release of Biomolecules from the Biomaterial Delivery Vehicles

Biomolecules or biomolecule combinations are identified for selective recruitment and proliferation of multipotent MSC, in particular, SMSC and BMSC, but not of macrophages. Inhibition of chondrogenesis by macrophages has been demonstrated in in vitro culture⁶³. SMSC and BMSC are harvested and expanded to passage 2 using established protocols. A panel of monoclonal antibodies are used to generate an antigen expression profile for the expanded MSC. Some of the candidate biomolecules to be screened for MSC mobilization, chemotaxis, proliferation, and differentiation include, but are not limited, to those listed in Table 3.

TABLE 3
Biomolecules for MSC mobilization, chemotaxis,
proliferation, and differentiation.
Biomolecules

Hepatocyte growth factor (HGF)
Stromal cell-derived factor (SDF-1)
Transforming growth factor beta-1, 3 (TGF beta-1, 3)
Bone morphogenetic protein 2, 4, 7 (BMP 2, 4, 7)
Platelet derived growth factor-bb (PDGF-bb)
Basic fibroblast growth factor (bFGF)
Insulin-like growth factor -I, -II (IGF-I, IGF-II) &
Insulin-like growth factor binding protein -3, -5 (IGFBP-3, -5)
Interleukin-8 (IL-8)
Interleukin-10 (IL-10)

The starting point for the proliferation assay is the testing for cells that migrated in response to the selected chemotaxis biomolecule. Mobilization biomolecules are screened using synovium explants, a chemotactic factor, and a chemotaxis chamber. For the chemotaxis, mobilization, and proliferation testing, the biomolecule(s) that most improve the yield of multipotent MSC are chosen for in vivo testing. For the chondrogenesis assay, the biomolecules that result in the highest expression of chondrogenic markers, and not hypertrophic markers, are selected for further use.

In addition, biomaterials are selected for their biocompatibility and biomolecule delivery characteristics. Initially, three delivery vehicle types are fabricated, nano-particles for initial mobilization cues, a gelatin-chitosan scaffold for neo tissue support and long-term differentiation cues, and a soft hydrogel to infiltrate the scaffold pores to deliver chemotaxis and proliferation cues. Biomolecule releases are measured at selected time points. Biomolecule release profiles are iteratively determined by varying material type, crosslink density, and manner of biomolecule loading (e.g., incorporation in material, heparin binding, etc.).

### Example 10

### In Vivo Testing of the Effectiveness of Temporal Delivery of Biomolecules for Regenerating Articular Cartilage in a Rabbit Femoral Intercondylar Groove Defect Model

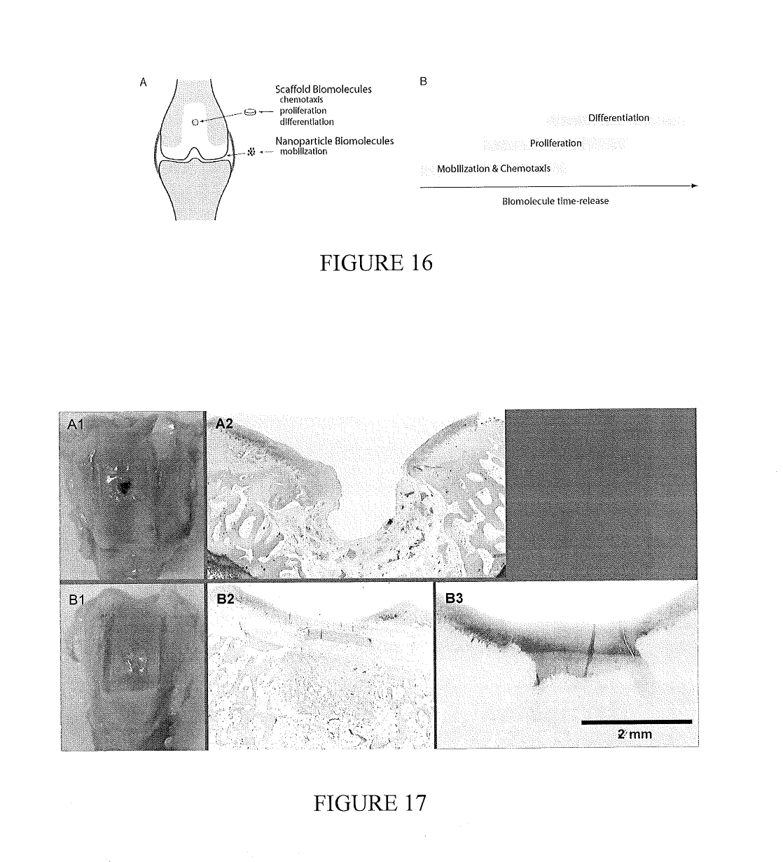

The well established⁶⁴⁻⁶⁶ rabbit femoral intercondylar groove defect model is used to evaluate cartilage regeneration. A cross-linked chitosan-gelatin scaffold is loaded with the differentiation biomolecule(s). The scaffold pores are infiltrated with a soft hydrogel containing the chemotaxis and proliferation biomolecules. Nanoparticles are injected into the joint space for short-term delivery of biomolecules to mobilize SMSC (FIGS. 16A, 16B). At 6 and 12 wk post surgery, defect healing is evaluated by macro-observation, micro-CT, and histologic staining.

### Example 11

### Nanoparticle Fabrication and Biomolecule Loading

Biomolecule-loaded poly(lactic-co-glycolic acid) (PLGA) nanoparticles are prepared by a water-in-oil method combining the vigorous sonication and low temperature slow emulsion as described^{56,57}. The particle size range is characterized and the biomolecule release profile is measured. The release goal is 10 ng per day from each injection of 100 uL of degradable particles. The decay of the release profile is important because the mobilization signal is intended to be short-term, approximately one week.

### Example 12

### Elastic Scaffold Fabrication and Biomolecule Loading

Biomolecule delivery from the composite scaffold is designed to have 2 main stages (FIG. 16B). A hybrid scaffold with 5% gelatin and 5% photocurable chitosan with 0.5% Iragure 2959 is used to fill the osteochondral defect³⁶. The scaffold is cured in a cylindrical mold with a 3.5 mm diameter and 3 mm height. After sterilization, heparin is immobilized on the surface to bind biomolecules that promote chondrogenic differentiation. The delivery goal is 10 ng per day per-biomolecule. Delivery is optimized to ramp up to the target level by day 7 and persist for at least 4 wk. An HA-gelatin based hydrogel is penetrated into the chitosan-gelatin scaffolds for delivery of chemotaxis and proliferation factors. The delivery goal is 10 ng per day per biomolecule. Delivery is optimized to ramp up to the target level by day 3 and persist for at least 2 wk. Additional parameters to consider include possible heparin binding of biomolecules and the cross-link density of the HA-gelatin hydrogel. Scaffolds are incubated in PBS at 37° C. for release profile testing.

### Example 13

### Animal Surgical Procedures and Tissue Collection

All animal procedures are conducted in strict compliance with protocols approved by the institutional animal care and use committee (IACUC). Skeletally mature (about age 8 mo) male New Zealand white rabbits are used for the in vivo studies. Surgery is performed using standard aseptic techniques. The distal femoral joint surface of the right knee is exposed through a 2.5 cm longitudinal anteromedial incision. A drill hole (3 mm diameter, 3 mm depth) is created in the femoral intercondylar groove. The scaffold is deformed to form a press fit in the defect. Nanoparticles are injected into the joint space immediately post surgery. There are a total of 8 treatment groups and 1 control group (Table 4). Each group consists of 7 animals for a total of 126 (9×7×2) rabbits and are evaluated at 6 and 12 wk post surgery. Ten additional rabbits are sacrificed for collection of bone marrow and knee synovial membrane.

TABLE 4
Experimental group assignments.

| Group | Hydrogel Infiltrate | Scaffold | Nanoparticles |
|---|---|---|---|
| No. | Chemotaxis/Proliferation | Differentiation | Mobilization |
| 1 | 1 | 1 | − |
| 2 | 1 | 1 | + |
| 3 | 1 | 2 | − |
| 4 | 1 | 2 | + |
| 5 | 2 | 1 | − |
| 6 | 2 | 1 | + |
| 7 | 2 | 2 | − |
| 8 | 2 | 2 | + |

| | | |
|---|---|---|
| 9 | Plain scaffold | Plain nanoparticles |

### Example 14

### In Vitro and In Vivo Experiments

For the in vitro experiments, each test is performed in triplicate to confirm the results. The antigen expression pattern of the expanded cell populations are characterized using a panel of monoclonal antibodies. Expression for each antigen is represented as a population proportion. Chemotaxis results are quantitated by the total number and types of cells recruited. Proliferation is quantified by the percent increase in cell number, the number of cell divisions, and the antigen profile. Chondrogenesis is evaluated by the expression levels of a selected panel of genes. Expression levels are normalized by the expression of GAPDH, a house-keeping gene.

For the in vivo experiments, the healing outcome are observed by macro photography and histological staining. Micro-CT images are used to measure the regeneration of subchondral bone as a proportion of the total defect area. A semi-quantitative score^{24,27} is assigned based on macroscopic, histological, and micro-CT results. To reduce inter-subject variation, the animal age and weight and the implant procedures is strictly controlled.

### Example 15

### Methods for Taking Measurements

Particle size. Particle size is examined using a high-resolution scanning electron microscope (Hitachi, Japan). The size distribution is quantified using ImagePro software.
Biomolecule release kinetics. Biomolecule release kinetics is measured using the enzyme-linked immunosorbant assay or high performance liquid chromatography.
Cell antigen expression. Cell antigen expression profiles is evaluated using flow cytometry and a panel of monoclonal antibodies. MSC are identified by an antigen expression pattern. The panel of monoclonal antibodies includes, but is not limited to, CD 14, CD44, CD45, CD73 (SH3, SH4), CD90, CD105 (SH2), and CD11b. Antibodies to assess multipotency includes, but is not limited to, CD166 (SB10, ALCAM)⁷¹, CD49a⁷², and Stro-1⁷³. Flow cytometry is performed with a FACSCalibur instrument and data is analyzed using CellQuest Pro software.
Chemotaxis. Chemotaxis of isolated cells is measured using a dual-compartment chamber consisting of a 24-well tissue culture plate and a Costar Transwell insert¹⁸ with a polycarbonate membrane filter (8 μm pores). The bottom of each well is covered with a thin layer (200 d) of cross-linked thiol-modified hyaluronin-gelatin hydrogel containing the test biomolecule. The hydrogel is covered with 400 μl of culture media; 10⁴ cells in 200 μl media is added to the insert. After incubation for 8 h at 37° C. and 5% CO₂, the inserts are removed from the wells and the cells are fixed and stained. The migrated cells are imaged using confocal laser microscopy and the cells are counted using ImagePro software. The nuclear stain DRAQ5 indicate the total cell number. Stro-1, CD166, and CD49a antibodies are used to assess multipotency of migrated cells. Wells with no biomolecule delivery serve as controls. Wells without inserts are used to measure biomolecule release at selected intervals. After optimization for chemotaxis has been completed, migrated cells are collected for the proliferation assay.
Mobilization. Mobilization of cells from synovial membrane explants are evaluated using the chemotaxis procedure. The selected chemotaxis biomolecule are used in the hydrogel. The mobilization test biomolecule is added to the culture medium. The explants are positioned on the membrane surface. The incubation period is empirically determined. Wells without the mobilization biomolecule serve as controls.
Cell proliferation. Cell proliferation are measured using the Click-iT® EdU assay that detects new DNA synthesis (Invitrogen). Proliferation is evaluated for cells cultured with 5 or 20 ng/ml biomolecule concentration. Because the assay is compatible with flow cytometry, the MSC antigen expression panel is used concurrently to determine if a biomolecule stimulates proliferation equally among the different MSC lineages. Cells are stained with PKH-26 at the beginning of the assay. Because the amount of dye is reduced by half at each cell division, the number of divisions for each cell type can also be measured.
Gene expression. Gene expression analysis is used to evaluate chondrogenic differentiation. BMSC and SMSC are induced down the chondrogenic pathway using the standard pellet culture protocol^{54,70}. Gene expression of the chondrogenic markers aggrecan, sox9, collagen II, syndecan-3 (early), and annexin VI⁷⁴ (late) are evaluated. Hypertrophic markers MMP 13 and col X are also be evaluated. For RT-PCR, total RNA is purified from cell pellets using the High Pure RNA isolation kit (Roche). The TaqMan RNA-to-C_{T} 2-step kit (Applied Biosystems) is used to reverse transcribe RNA to cDNA and then perform qPCR amplification using the—TaqMan Gene Expression Master Mix. Oligonucleotide primers for PCR amplification correspond to the gene expression profile selected for each of the differentiation assays.
Characterization of defect healing. Characterization of defect healing is performed six weeks and twelve weeks post surgery. The animals are sacrificed and the implant site is photographed and collected. μCT scans are acquired to evaluate the extent of subchondral bone regeneration. Regeneration is measured as a percentage of the total defect area. Histological sections are stained with H&E, Safranin O, and for collagens type I and II. Histological features, including GAG staining, surface smoothness, columnar alignment of chondrocytes, and regeneration of subchondral bone, are evaluated. An ordinal composite score is assigned^{24,27}.

### Example 16

### Methods for Data Management and Analysis

Quantitative data for each group is represented by the mean and the standard error of the mean. One-way analysis of variance (ANOVA) is performed for hypothesis testing using SPSS 9.0 software (SPSS Inc. Chicago, Ill.). The ordinal histology scores is evaluated using logistic regression. Statistical significance is set at p<0.05.

### Example 17

### Estimation of Sample Size and Power

Sample size is estimated using the standard power analysis formula⁷⁵ where n=2[(Z_{α}−Z_{β})σ/(m₁−m₂)]². The standard deviation, σ, is assumed to be 15% and the significant difference (m₁-m₂) is assumed equal to or larger than 20%. The resulting group size is 6.64 (≈7) animals.

### Example 18

### Further Data on Scaffold Production and In Vivo Studies

Materials. High molecular weight chitosan with 85% deacetylation and 3-(3-dimethylaminopropyl)-1-ethylcarbodiimide hydrochloride (EDC) were purchased from TCI America (Portland, Oreg., USA). Methanesulfonic acid (98%) and methacryloyl chloride were purchased from Alfa Aesar (Ward Hill, Mass., USA). Benzoyl chloride, sodium chloride, heparin sodium, 2-(N-norpholino)ethanesulfonic acid (MES), and N-hydroxysuccinimide (NHS) were purchased from Sigma Aldrich (St. Louis, Mo., USA). Sodium hydroxide (5N) was purchased from VWR (West Chester, Pa., USA). Gelatin type A (100 Bloom strength) and dimethyl sulfoxide were purchased from Thermo Fisher Scientific (Waltham, Mass., USA). Irgacure® 2959 was kindly provided by Ciba Specialty Chemicals (Basal, Switzerland). All signaling molecules were purchased from Peprotech (Rocky Hill, N.J., USA). Antibodies for types I and II collagen were purchased from Abeam (Cambridge, Mass., USA) and Chondrex (kit, Redmond, Wash., USA) respectively. A tyramide signal amplification kit was purchased from Invitrogen (Carlsbad, Calif., USA).

Synthesis of photocurable chitosan. Chitosan was chemically modified to contain methacrylate groups for photo-initiator dependent curability and benzoic groups to improve solubility in organic solvents. The amino group is protected during the modification. Briefly, 1 g of chitosan was dissolved into 15 ml of methanesulfonic acid with continuous stirring for 25 min. A solution of 1.1 g benzoyl chloride and 1.227 g methacryloyl chloride was added dropwise, and stirring continued for an additional 30 min. The photocurable chitosan was precipitated by adding the chitosan-acid solution dropwise to an aqueous solution of ammonium hydroxide (100 ml 5N sodium hydroxide+600 ml DI water) with gentle stirring. The precipitate was washed 10 times with DI water to remove reagent and solvent residues and was dried under vacuum overnight.
Scaffold fabrication. A 5% gelatin-7.5% photocurable chitosan scaffold was prepared for in vivo testing. A 5% (w/w) gelatin-DMSO solution was prepared under constant stirring. Photocurable chitosan was added to the gelatin-DMSO solution to produce a 7.5% (per weight DMSO) chitosan solution. Irgacure 2959 was then added at 0.5% (per weight chitosan).

Scaffolds were prepared using an 8 mm diameter mold set to a depth of 2 mm. The chitosan-gelatin solution was dropped into the mold. The chitosan was photopolymerized to form a cross-linked network by exposing the scaffold to light at 800 mW/cm² intensity and 365 nm wavelength for 3 min. The scaffold was pushed out of the mold and washed copiously in DI water to remove solvent residue. The scaffolds were trimmed to a diameter of 3.5 mm using a biopsy punch. The pore size of the scaffolds are 300-325 μm.

Signaling molecule binding. Scaffolds were heparinized to bind and protect signaling molecules for in vivo delivery. The heparin solution, 0.05 M MES buffer (pH 5.6) with 0.2% heparin sodium, 0.2% EDC, and 0.12% NHS (all % w/v), was incubated at 37° C. for 10 min to activate the heparin carboxyl groups. Scaffolds were immersed in the activated heparin solution (1 ml/scaffold) and placed under vacuum for 10 min to remove air bubbles. The scaffolds were incubated at 37° C. for 4 hr. To ensure the interior pores of the scaffold were heparinized, the scaffolds were blot dried and fresh heparin solution was dropped onto each scaffold. The scaffolds were incubated at 37° C. for an additional 4 hr. The scaffolds were then washed in 0.1 M Na₂HPO₄ for 2 hr, 4 M NaCl (4 times for 24 hr), and DI water (5 times for 24 hr). The scaffolds were sterilized in 75% ethanol. The EDC in the heparin binding reactions also crosslinked the gelatin and the gelatin to the chitosan.
Signaling molecules were bound to the scaffold by carefully pipetting 13 μl of solution onto each scaffold under sterile conditions. Control scaffolds were loaded with 1 ug of IGF-1 each. Treatment scaffolds were loaded with 1 ug each of IGF-1, IGF-BP2, TGFB-1, IL-10, HGF, and FGF-basic. The scaffolds were incubated with the signaling molecule solution overnight.
In vivo evaluation of cartilage healing. Osteochondral regeneration was evaluated in the knee patellar groove of 3-month male New Zealand white rabbits. The patellar groove was exposed using a medial para-patellar incision and lateral displacement of the patella. A 3 mm diameter and 2 mm deep osteochondral defect was created in the patellar groove using a trephine drill bit. The 3.5 mm diameter scaffold was press-fit in place, the patella was repositioned, and the incision was sutured in layers. There were a total of 20 rabbits, 10 treatment and 10 control. Five rabbits from each group were sacrificed at 6 and 12 weeks post surgery. The distal femur was fixed in neutral buffered formalin, decalcified, embedded in paraffin, and sectioned in accordance with standard histology protocols. Sections were stained for glycosaminoglycan content using Safranin 0 fast green. Immunohistochemistry was used to evaluate cartilage collagen type 1 and 2 distribution. Tyramide signal amplification was used to better visualize the distribution of type 2 collagen

In vivo evaluation of cartilage healing in a critical-sized osteo-chondral defect was done to demonstrate the hyaline cartilage regeneration in our highly elastic scaffold loaded with a biomolecule cocktail. The treatment group received scaffolds loaded with a biomolecule mixture consisting of TGFb-1, IL-10, IGF-1, and IGF binding protein 2, HGF, and bFGF. The control group received scaffolds loaded with IGF-1 only. Six weeks post surgery, the treatment group exhibited hyaline cartilage healing and regeneration (FIG. 17B1, FIG. 17 B2, and FIG. 17 B3), while the control group did not (FIG. 17A1-A2). The cartilage had a columnar cell organization and stained intensely with Safranin 0 through the full thickness, indicating the presence of glycosaminoglycan (GAG).

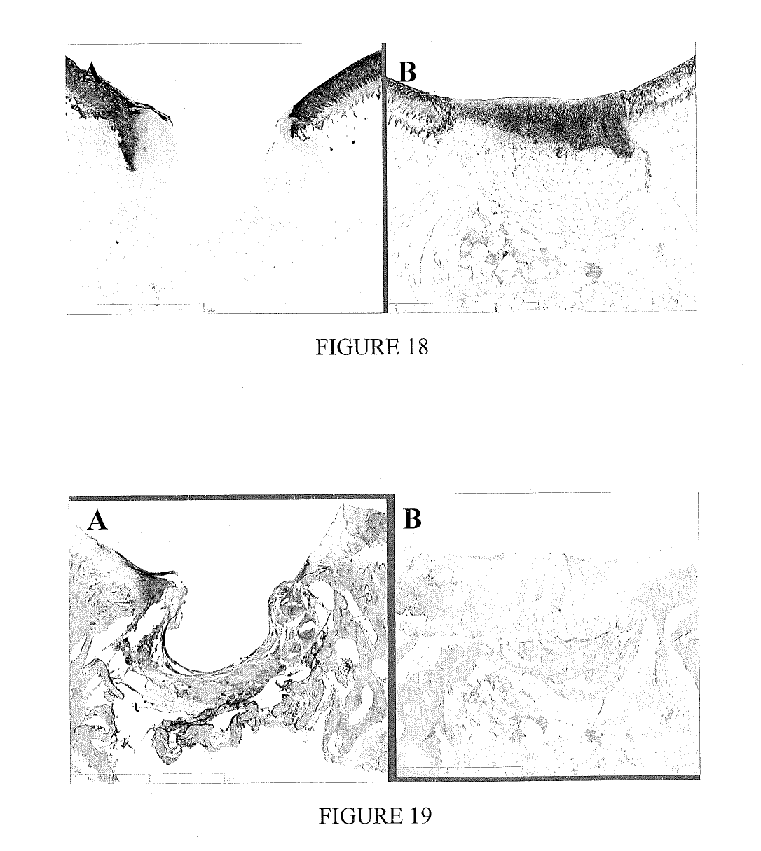

Immunostaining for collagen type I and II, as shown in FIG. 18 and FIG. 19, further demonstrate hyaline cartilage regeneration at the lesion site (FIG. 18B, dark stain). For treatment group, the cartilage regeneration site and intact cartilage site express collagen type II (FIG. 18B, dark stain). In the control group, the lesion site lacks collagen type II expression (FIG. 18A). Collagen type I immunostain as shown in FIG. 19 demonstrates that in the treatment group, the cartilage regeneration site and intact cartilage site do not express collagen type I (FIG. 19B). The underlying bone tissue expresses collagen type I (dark stain). The control group shows the lesion site with scar tissue expressing collagen type I (FIG. 19A).

The above examples clearly illustrate the advantage of the invention. Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference into this application in order to more fully describe the state of the art to which this invention pertains.

### REFERENCES

1. Schmitt, C., Sanchez, C., Desobry-Banon, S. & Hardy, J. Structure and technofunctional properties of protein-polysaccharide complexes: a review. Crit Rev Food Sci Nutr 38, 689-753 (1998).
2. Zeman, L. & Patterson, D. Effects of the solvent on polymer incompatibility in solution. Macromolecules 5, 513 (1972).
3. Dickinson, E. Colloidal aspects of beverages. Food Chemistry 51, 343-347 (1994).
4. Piculell, L., Bergfeldt, K. & Nilsson, S. in Biopolymer mixtures (eds. Harding, S., Hill, S. E. & Mitchell, J. R.) 13-36 (Nottingham University Press, Nottingham, 1995).
5. Qiu, Y., Zhang, N., Kang, Q., An, Y. & Wen, X. Fabrication of permeable tubular constructs from chemically modified chitosan with enhanced antithrombogenic property. Journal of Biomedical Materials Research: Part B (In press).
6. Gupta, A. N., Bohidar, H. B. & Aswal, V. K. Surface patch binding induced intermolecular complexation and phase separation in aqueous solutions of similarly charged gelatin-chitosan molecules. Journal of Physical Chemistry B 111, 10137-10145 (2007).
7. Perng, C. K. et al. Culturing adult human bone marrow stem cells on gelatin scaffold with pNIPAAm as transplanted grafts for skin regeneration. J Biomed Mater Res A (2007).
8. Yamamoto, M., Takahashi, Y. & Tabata, Y. Controlled release by biodegradable hydrogels enhances the ectopic bone formation of bone morphogenetic protein. Biomaterials 24, 4375-83 (2003).
9. Mutalik, V. et al. Aqueous-solution and solid-film properties of poly(vinyl alcohol), poly(vinyl pyrrolidone), gelatin, starch, and carboxymethylcellulose polymers. Journal of Applied Polymer Science 106, 765-774 (2007).
10. Ma, L., Gao, C. & Shen, J. [Factors controlling the microstructure of collagen-based dermis regeneration scaffold]. Sheng Wu Yi Xue Gong Cheng Xue Za Zhi 21, 311-5 (2004).
11. Gurtner, G. C., Callaghan, M. J. & Longaker, M. T. Progress and potential for regenerative medicine. Annu Rev Med 58, 299-312 (2007).
12. Rosso, F. et al. Smart materials as scaffolds for tissue engineering. J Cell Physiol 203, 465-70 (2005).
13. Stevens, M. M. & George, J. H. Exploring and engineering the cell surface interface. Science 310, 1135-8 (2005).
14. Guo, T. et al. Porous chitosan-gelatin scaffold containing plasmid DNA encoding transforming growth factor-beta1 for chondrocytes proliferation. Biomaterials 27, 1095-103 (2006).
15. Huang, Y., Onyeri, S., Siewe, M., Moshfeghian, A. & Madihally, S. V. In vitro characterization of chitosan-gelatin scaffolds for tissue engineering. Biomaterials 26, 7616-27 (2005).
16. Li, K. et al. Chitosan/gelatin composite microcarrier for hepatocyte culture. Biotechnol Lett 26, 879-83 (2004).
17. Kim, H. W., Knowles, J. C. & Kim, H. E. Porous scaffolds of gelatin-hydroxyapatite nanocomposites obtained by biomimetic approach: characterization and antibiotic drug release. J Biomed Mater Res B Appl Biomater 74, 686-98 (2005).
18. Woo, K. M. et al. Nano-fibrous scaffolding promotes osteoblast differentiation and biomineralization. Biomaterials 28, 335-43 (2007).
19. Hartgerink, J. D., Beniash, E. & Stupp, S. I. Self-assembly and mineralization of peptide-amphiphile nanofibers. Science 294, 1684-8 (2001).
20. Li, W. J., Laurencin, C. T., Caterson, E. J., Tuan, R. S. & Ko, F. K. Electrospun nanofibrous structure: a novel scaffold for tissue engineering. J Biomed Mater Res 60, 613-21 (2002).
21. Shin, M., Yoshimoto, H. & Vacanti, J. P. In vivo bone tissue engineering using mesenchymal stem cells on a novel electrospun nanofibrous scaffold. Tissue Eng 10, 33-41 (2004).
22. Gibson, L. J. & Ashby, M. E. in Cellular Solids: Structure and Properties (second edition) (eds. Gibson, L. J. & Ashby, M. E.) 175-234 (Cambridge University Press, Cambridge, UK, 1997).
23. Qiu, Y., Cui, L., Kang, Q. & Wen, X. Synthesis and Characterization of Novel Light curable chitosans for Bone Tissue Engineering. Biomacromolecules (submitted).
1 Lawrence, R. C, et al., Estimates of the prevalence of arthritis and other rheumatic conditions in the United States. Part II. Arthritis Rheum 58 (1), 26 (2008).
2 American Academy of Orthopaedic Surgeons: Physician visits by patients with arthritis, Available at http://www.aaos.org/research/stats/Arthritis.pdf, (2009).
3 American Academy of Orthopaedic Surgerons: Facts on knee replacements, Available at http://www.aaos.org/research/stats/Knee_Facts.pdf, (2009).
4 American Academy of Orthopaedic Surgerons: Facts on hip replacements, Available at http://www.aaos.org/research/stats/Hip_Facts.pdf, (2009).
5 Kurtz, S. et al., Projections of primary and revision hip and knee arthroplasty in the United States from 2005 to 2030. J Bone Joint Surg Am 89 (4), 780 (2007).
6 Brittberg, M. et al., Treatment of deep cartilage defects in the knee with autologous chondrocyte transplantation. N Engl J Med 331 (14), 889 (1994).
7 Steck, E. et al., Mesenchymal stem cell differentiation in an experimental cartilage defect: restriction of hypertrophy to bone-close neocartilage. Stem Cells DeV (2008).
8 Genzyme Corporation Carticel Product Label, Available at http://www.carticel.com/pdfs/carticel_product_label.pdf, (2007).
9 Williams, R. J., 3rd and Harnly, H. W., Microfracture: indications, technique, and results. Instr Course Lect 56, 419 (2007).
10 Horas, U. et al., Autologous chondrocyte implantation and osteochondral cylinder transplantation in cartilage repair of the knee joint. A prospective, comparative trial. J Bone Joint Surg Am 85-A (2), 185 (2003).
11 Stoop, R., Smart biomaterials for tissue engineering of cartilage. Injury 39 Suppl 1, S77 (2008).
12 Sohier; J. et al., Critical factors in the design of growth factor releasing scaffolds for cartilage tissue engineering. Expert Opin Drug Deliv 5 (5), 543 (2008).
13 Stayton, P. S. et al., ‘Smart’ delivery systems for biomolecular therapeutics. Orthod Craniofac Res 8 (3), 219 (2005).
14 Frenkel, S. R. and Di Cesare, P. E., Scaffolds for articular cartilage repair. Ann Biomed Eng 32 (1), 26 (2004).
15 Miljkovic, N.D., Cooper, G. M., and Marra, K. G., Condrogenesis, bone morphogenetic protien-4 and mesenchymal stem cells. Osteoarthritis Cartilage (2008).
16 Kurth, T. et al., Chondrogenic potential of human synovial mesenchymal stem cells in alginate. Osteoarthritis Cartilage 15 (10), 1178 (2007).
17 Lorenz, H. P. and Longaker, M. T., in Essential Practice of Surgery (Springer, N.Y., 2003), pp. 77.
18 Zhao, J., Zhang, N, Prestwich, G. D., and Wen, X., Recruitment of Endogenous Stem Cells for Tissue Repair. Macromol Biosci (2008).
19 Cianciolo, G. et al., PDGF-AB release during and after haemodialysis procedure. Nephrol Dial Transplant 14 (10), 2413 (1999).
20 Breinan, H. A. et al., Effect of cultured autologous chondrocytes on repair of chondral defects in a canine model. J Bone Joint Surg Am 79 (10), 1439 (1997).
21 Brittberg, M. et al., Rabbit articular cartilage defects treated with autologous cultured chondrocytes. Clin Orthop Relat Res (326), 270 (1996).
22 Nehrer, S., Spector, M., and Minas, T., Histologic analysis of tissue after failed cartilage repair procedures. Clin Orthop Relat Res (365), 149 (1999).
23 Sittinger, M. and Burmester, G. R., Can engineered cartilage transplants be used for treating rheumatic diseases? Nat Clin Pract Rheumatol 2 (4), 172 (2006).
24 Gotterbarm, T. et al., An in vivo study of a growth-factor enhanced, cell free, two-layered collagen-tricalcium phosphate in deep osteochondral defects. Biomaterials 27 (18), 3387 (2006).
25 Schagemann, J. C. et al., Cell-Laden and Cell-Free Biopolymer Hydrogel for the Treatment of Osteochondral Defects in a Sheep Model. Tissue Eng Part A 15 (1), 75 (2009).
26 Erggelet, C. et al., Regeneration of ovine articular cartilage defects by cell-free polymer-based implants. Biomaterials 28 (36), 5570 (2007).
27 Tamai, N. et al., A new biotechnology for articular cartilage repair: subchondral implantation of a composite of interconnected porous hydroxyapatite, synthetic polymer (PLA-PEG), and bone morphogenetic protein-2 (rhBMP-2). Osteoarthritis Cartilage 13 (5), 405 (2005).
28 Brittberg, M. and Lindahl, A., in Tissue Engineering, edited by C. van Blitterswijk (Academic Press, Boston, 2008).
29 Kuo, C. K., Li, W. J., Mauck, R. L., and Tuan, R. S., Cartilage tissue engineering: its potential and uses. Curr Opin Rheumatol 18 (1), 64 (2006).
30 Ponticiello, M. S., Schinagl, R. M., Kadiyala, S., and Barry, F. P., Gelatin-based resorbable sponge as a carrier matrix for human mesenchymal stem cells in cartilage regeneration therapy. J Biomed Mater Res 52 (2), 246 (2000).
31 Awad, H. A. et al., Chondrogenic differentiation of adipose-derived adult stem cells in agarose, alginate, and gelatin scaffolds. Biomaterials 25 (16), 3211 (2004).
32 Angele, P. et al., Characterization of esterified hyaluronan-gelatin polymer composites suitable for chondrogenic differentiation of mesenchymal stem cells. J Biomed Mater Res A (2008).
33 Brown, C. D. and Hoffman, A. S., in Methods of tissue engineering, edited by A. Atala and R. P. Lanza (Academic Press, San Diego, 2002), pp. 1285.
34 Di Martino, A., Sittinger, M., and Risbud, M. V., Chitosan: a versatile biopolymer for orthopaedic tissue-engineering. Biomaterials 26 (30), 5983 (2005).
35 Gomes, M. et al., in Tissue engineering, edited by C. van Blitterswijk (Academic Press, Boston, 2008), pp. 740.
36 Qui, Y., Zhang, N., and Wen, X., Controlling the microstructure of hybrid scaffolds by governing transient phase separation in polysaccharide-protein-organic solvent system. Manuscript under review (2009).
37 Lutolf, M. P. et al., Repair of bone defects using synthetic mimetics of collagenous extracellular matrices. Nat Biotechnol 21 (5), 513 (2003).
38 Cai, S., Liu, Y., Zheng Shu, X., and Prestwich, G. D., Injectable glycosaminoglycan hydrogels for controlled release of human basic fibroblast growth factor. Biomaterials 26 (30), 6054 (2005).
39 Pike, D. B. et al., Heparin-regulated release of growth factors in vitro and angiogenic response in vivo to implanted hyaluronan hydrogels containing VEGF and bFGF. Biomaterials 27 (30), 5242 (2006).
40 Baksh, D., Song, L., and Tuan, R. S., Adult mesenchymal stem cells: characterization, differentiation, and application in cell and gene therapy. J Cell Mol Med 8 (3), 301 (2004).
41 Tuan, R. S., Boland, G., and Tuli, R., Adult mesenchymal stem cells and cell-based tissue engineering. Arthritis Res Ther 5 (1), 32 (2003).
42 De Bari, C., Dell'Accio, F., Tylzanowski, P., and Luyten, F. P., Multipotent mesenchymal stem cells from adult human synovial membrane. Arthritis Rheum 44 (8), 1928 (2001).
43 Sakaguchi, Y., Sekiya, I., Yagishita, K., and Muneta, T., Comparison of human stem cells derived from various mesenchymal tissues: superiority of synovium as a cell source. Arthritis Rheum 52 (8), 2521 (2005).
44 Mochizuki, T. et al., Higher chondrogenic potential of fibrous synovium- and adipose synovium-derived cells compared with subcutaneous fat-derived cells: distinguishing properties of mesenchymal stem cells in humans. Arthritis Rheum 54 (3), 843 (2006).
45 Tsutsumi, S. et al., Retention of multilineage differentiation potential of mesenchymal cells during proliferation in response to FGF. Biochem Biophys Res Commun 288 (2), 413 (2001).
46 Fiedler, J., Brill, C., Blum, W. F., and Brenner, R. E., IGF-I and IGF-II stimulate directed cell migration of bone-marrow-derived human mesenchymal progenitor cells. Biochem Biophys Res Commun 345 (3), 1177 (2006).
47 Neuss, S. et al., Functional expression of HGF and HGF receptor/c-met in adult human mesenchymal stem cells suggests a role in cell mobilization, tissue repair, and wound healing. Stem Cells 22 (3), 405 (2004).
48 Zhong, R. et al., Small peptide analogs to stromal derived factor-1 enhance. chemotactic migration of human and mouse hematopoietic cells. Exp Hematol 32 (5), 470 (2004).
49 Scherping, S. C., Jr. et al., Effect of growth factors on the proliferation of ligament fibroblasts from skeletally mature rabbits. Connect Tissue Res 36 (1), 1 (1997).
50 Avdiushko, R. et al., IL-10 receptor dysfunction in macrophages during chronic inflammation. J Leukoc Biol 70 (4), 624 (2001).
51 Ringe, J. et al., Towards in situ tissue repair: human mesenchymal stem cells express chemokine receptors CXCR1, CXCR2 and CCR2, and migrate upon stimulation with CXCL8 but not CCL2. J Cell Biochem 101 (1), 135 (2007).
52 An, H. Y. and Martin, K. L., Handbook of histology methods for bone and cartilage. (Humana Press, Totowa, N.J., 2003).
53 An, H. Y. and Friedman, R. J., Animal Models in orthopaedic research. (CRC Press, Inc., Boca Raton, Fla., 1999).
54 Reger, R. L., Tucker, A. H., and Wolfe, M. R., in Mesenchymal stem cells methods and protocols, edited by D. J. Prockop, B. A. Bunnell, and D. G. Phinney (Springer, 2008), Vol. 449, pp. 93.
55 Zhang N, Wright G, and X J., Wen, presented at the American Chemical Society 2005 (unpublished).
56 Wright, G. et al., Induction of in vivo angiogenesis achieved by activation the cellular oxygen-sensing pathway through biocompatible degradable carrier release of prolyl hydroxylase inhibitors. Biotechnology and Bioengineering (Under review).
57 Zhang, C., Zhang, N., and Wen, X., Synthesis and characterization of biocompatible, degradable, light-curable, polyurethane-based elastic hydrogels. J Biomed Mater Res A 82 (3), 637 (2007).
58 Matsuda-Hashii, Y. et al., Hepatocyte growth factor plays roles in the induction and autocrine maintenance of bone marrow stromal cell IL-11, SDF-1alpha, and stem cell factor. Exp Hematol 32 (10), 955 (2004).
59 Fujii, K. et al., Elevation of serum hepatocyte growth factor during granulocyte colony-stimulating factor-induced peripheral blood stem cell mobilization. Br J Haematol 124 (2), 190 (2004).
60 Weimar, I. S. et al., Hepatocyte growth factor/scatter factor (HGF/SF) is produced by human bone marrow stromal cells and promotes proliferation, adhesion and survival of human hematopoietic progenitor cells (CD34+). Exp Hematol 26 (9), 885 (1998).
61 Takai, K. et al., Hepatocyte growth factor is constitutively produced by human bone marrow stromal cells and indirectly promotes hematopoiesis. Blood 89 (5), 1560 (1997).
62 Simmons P J, Torok-Storb B., Identification of stromal cell precursors in human bone marrow by a novel monoclonal antibody, STRO-1. Blood. 78 (1), 55 (1991).
63 Pei, M., He, F., Kish, V. L., and Vunjak-Novakovic, G., Engineering of functional cartilage tissue using stem cells from synovial lining: a preliminary study. Clin Orthop Relat Res 466 (8), 1880 (2008).
64 An, H. Y. and Friedman, R. J., in Animal Models in Orthopaedic Research, edited by H. Y. An and R. J. Friedman (CRC Press, Boca Raton, Fla., 1999), pp. 309.
65 Lee, D. K., Kang, Q. K., Gutowska, A., and al, et, Poly-n-acetyl Glucosamine-SO4 for repairing osteochondral defect in rabbits. Key Engineering Materials 288-289, 83 (2004).
66 Lee, D. K., Kang, Q. K., Gutowska, A., and al, et, Repair of rabbit cartilage defects using hydrogels and scaffolds seeded with autologous chondrocyte—a preliminary report. USC Orthopaedic Journal 1, 35 (2002).
67 Hunziker, E. B. and Rosenberg, L. C., Repair of partial-thickness defects in articular cartilage: cell recruitment from the synovial membrane. J Bone Joint Surg Am 78 (5), 721 (1996).
68 Shirasawa, S, et al., In vitro chondrogenesis of human synovium-derived mesenchymal stein cells: optimal condition and comparison with bone marrow-derived cells. J Cell Biochem 97 (1), 84 (2006).
69 Fiedler, J., Roderer, G., Gunther, K. P., and Brenner, R. E., BMP-2, BMP-4, and PDGF-bb stimulate chemotactic migration of primary human mesenchymal progenitor cells. J Cell Biochem 87 (3), 305 (2002).
70 Nimura, A. et al., Increased proliferation of human synovial mesenchymal stem cells with autologous human serum: comparisons with bone marrow mesenchymal stem cells and with fetal bovine serum. Arthritis Rheum 58 (2), 501 (2008).
71 Stewart, K. et al., STRO-1, HOP-26 (CD63), CD49a and SB-10 (CD166) as markers of primitive human marrow stromal cells and their more differentiated progeny: a comparative investigation in vitro. Cell Tissue Res 313 (3), 281 (2003).
72 Rider, D. A., Nalathamby, T., Nurcombe, V., and Cool, S. M., Selection using the alpha-1 integrin (CD49a) enhances the multipotentiality of the mesenchymal stem cell population from heterogeneous bone marrow stromal cells. J Mol Histol 38 (5), 449 (2007).
73 Gronthos, S. et al., Molecular and cellular characterisation of highly purified stromal stem cells derived from human bone marrow. J Cell Sci 116 (Pt 9), 1827 (2003).
74 Pfander, D., Swoboda, B., and Kirsch, T., Expression of early and late differentiation markers (proliferating cell nuclear antigen, syndecan-3, annexin VI, and alkaline phosphatase) by human osteoarthritic chondrocytes. Am J Pathol 159 (5), 1777 (2001).
75 Dawson-Saunders, B. and Trapp, R., Basic and clinical biostatistics. (Appleton & Lange, 1990).

1. A biocompatible, biodegradable, three-dimensional scaffold comprising a photocurable polysaccharide and a protein.

2. The scaffold of claim 1, wherein the photocurable polysaccharide is photocurable chitosan.

3. The scaffold of claim 1, wherein the protein is gelatin.

4. The scaffold of claim 2, wherein the photocurable chitosan comprises benzoic groups and methacrylate groups substituted for the chitosan side chain hydroxyl groups.

5. A biocompatible, biodegradable, elastic cell free scaffold comprising at least one biomolecule bound directly to the scaffold.

6. The scaffold of claim 1, wherein at least one biomolecule is bound directly to the scaffold.

7. The scaffold of claim 6, wherein at least one biomolecule is bound to the scaffold through heparin.

8. The scaffold of claim 6, wherein the biomolecule is a differentiation stimulating biomolecule selected from the group consisting of: a bone morphogenic protein (BMP), a transforming growth factor (TGF), an insulin-like growth factor, and any combination thereof.

9. The scaffold of claim 8, wherein the bone morphogenic protein (BMP) is selected from the group consisting of BMP-2, BMP-4, BMP-5, BMP-6, BMP-7, and any combination thereof.

10. The scaffold of claim 8, wherein the transforming growth factor (TGF) is selected from the group consisting of TGF β-1, TGF β-3, and any combination thereof.

11. The scaffold of claim 8, wherein the insulin-like growth factor is insulin-like growth factor-1.

12. The scaffold of claim 6, wherein the biomolecule is a chemotaxis and/or proliferation stimulating biomolecule selected from the group consisting of hepatocyte growth factor (HGF), stromal cell-derived growth factor-1 (SDF-1), platelet derived growth factor-bb (PDGF-bb), insulin-like growth factor (IGF), insulin-like growth factor binding protein (IGFBP), interleukin and any combination thereof.

13-15. (canceled)

16. The scaffold of claim 6, wherein the biomolecule is a mobilization stimulating biomolecule.

17. The scaffold of claim 16, wherein the biomolecule is hepatocyte growth factor (HGF).

18. The scaffold of claim 1, further comprising a hydrogel comprising one or more biomolecules, singly or in any combination.

19. The scaffold of claim 18, wherein the hydrogel is a thiolated extracellular matrix selected from the group consisting of thiolated collagen, thiolated gelatin, thiolated laminin, thiolated fibronectin, thiolated heparin, and thiolated hyaluronan (HA), and any combination thereof.

20. (canceled)

21. The scaffold of claim 18, wherein the biomolecule is a chemotaxis and/or proliferation stimulating biomolecule for mesenchymal stem cells.

22. The scaffold of claim 18, wherein the biomolecule is a chemotaxis and/or proliferation biomolecule selected from the group consisting of hepatocyte growth factor (HGF), stromal cell-derived growth factor-1 (SDF-1), platelet derived growth factor-bb (PDGF-bb), insulin-like growth factor (IGF), insulin-like growth factor binding protein (IGFBP), interleukin and any combination thereof.

23-25. (canceled)

26. A method of producing a scaffold comprising photocurable polysaccharide and protein, comprising:
a) adding a photocurable polysaccharide in a solvent to a protein-solvent mixture to make a polysaccharide-protein-solvent mixture;
b) adding a photoinitiator to the mixture of step (a) above; and
c) exposing the polysaccharide-protein-DMSO mixture of step (b) to ultraviolet (UV) light to photocure the photocurable polysaccharide, whereby a scaffold comprising photocurable polysaccharide and protein is produced.

27. The method of claim 26, wherein the polysaccharide-protein-solvent mixture of step (b) is allowed to set for a period of time of zero hours to about five days at a temperature of about 10 degrees Celsius to about 60 degrees Celsius prior to exposure to the UV light.

28. The method of claim 26, wherein the photocurable polysaccharide is photocurable chitosan.

29. The method of claim 26, wherein the protein is gelatin.

30. The method of claim 28, wherein the concentration of the chitosan in the chitosan-protein-DMSO mixture is 5% (w/w/w) or 7.5% (w/w/w).

31. (canceled)

32. The method of claim 26, further comprising boiling the scaffold.

33. The method of claim 26, further comprising contacting the scaffold of step (c) with a solution of 1-ethyl-(3-3-dimethylaminopropyl carbodiimide hydrochloride) (EDC).

34. The method of claim 26, further comprising binding a biomolecule directly to the scaffold.

35. The method of claim 26, further comprising binding the biomolecule to the scaffold via a linking molecule.

36. A method of regenerating tissue in a subject, comprising contacting the subject with the scaffold of claim 1.

37. The method of claim 36, where the subject does not receive a cell transplant as part of the tissue regeneration process, either prior to, during or after contacting with the scaffold.

38. The method of claim 36, further comprising delivering nanoparticles and/or microspheres comprising at least one biomolecule to the subject.

39. (canceled)

40. A method of regenerating cartilage in a subject having a partial cartilage defect, a full thickness defect and/or an osteochondral defect, comprising contacting the defect(s) with the scaffold of claim 1.

41. The method of claim 40, further comprising delivering nanoparticles and/or microspheres comprising at least one biomolecule to the subject, wherein the delivery is directly into a joint space having the defect.

42. The method of claim 40, wherein the at least one biomolecule is a biomolecule that stimulates the mobilization of mesenchymal stem cells.

43. The method of claim 41, wherein the biomolecule is hepatocyte growth factor (HGF).

44. The method of claim 41, wherein the at least one biomolecule comprises TGF-β-1, IL-10, IGF-1, IGF binding protein 2, HGF, bFGF or any combination thereof.

45. The method of claim 36, wherein the scaffold is selected from the group consisting of: (1) chitosan and gelatin; (2) chitosan and collagen; (3) chitosan, collagen, and gelatin; (4) elastin; (5) elastin and collagen; (6) elastin and chitosan; (7) polyurethane; (8) poly(lactide-co-caprolactone); (9) poly(glycolide-co-caprolactone); (10) poly(1,8-octanediol citrate); (11) polydimethylsiloxane; (11) gelatin and poly(lactide-co-caprolactone); and (12) polyurea.

46. The method of claim 38, wherein the at least one biomolecule comprises a combination of biomolecules selected from the group consisting of the combination of biomolecules of: (1) IGF-1, IGF-BP2, TGFB-1, IL-10, HGF, and FGF-basic; (2) TGF-alpha, bFGF, PDGF, TGF beta-3, IL-8, IGF-1, and IGF-II; (3) SCF, SDF-1, IL-10, HB-EGF, IGF-1, and IGF-II; (4) TGF-alpha, PDGF-AB, TGF-beta-3, BMP-2, IL-10, and IGF-2; (5) HGF, SCF, PDGF-BB, IGF-1, IL-10, and TGF-beta-3; (6) HGF, TGF-alpha, PDGF-BB, IGF-1, IGF-2, IL-10, and TGF-beta-3; and (7) TGF-alpha, PDGF, TGF-beta-3, IL-10, IGF-1, and IGF-2.
